(19) United States
(12) Patent Application Publication
(10) Pub. No.: US 2009/0062143 A1
(43) Pub. Date: Mar. 5, 2009

(54) TRANSLATION INITIATION REGION SEQUENCES FOR OPTIMAL EXPRESSION OF HETEROLOGOUS PROTEINS

(75) Inventors: Thomas M. Ramseier, Newton, MA (US); Russell J. Coleman, San Diego, CA (US); Jane C. Schneider, San Diego, CA (US)

Correspondence Address: Alston & Bird LLP;Dow Global Technologies, Inc., Bank Of America Plaza, 101 South Tryon Street, Suite 4000, Charlotte, NC, 28280-4000, US

(73) Assignee: Dow Global Technologies Inc., Midland, MI (US)

(21) Appl. No.: 12/185,726

(22) Filed: Aug. 4, 2008

Related U.S. Application Data
(60) Provisional application No. 60/953,813, filed on Aug. 3, 2007.

(51) Int. Cl. C40B 30/06; C07H 21/00; C12N 15/63; C12N 1/00; C40B 40/06

(52) U.S. Cl. 506/10; 536/23.1; 435/320.1; 435/243; 506/16

(57) ABSTRACT

The present invention provides methods and compositions for producing heterologous protein with improved yield and/or quality. A library of randomized ribosomal binding site sequences is provided for the identification of a translation initiation region sequence optimal for expression of the heterologous protein. Also provided are novel ribosomal binding site sequences, and vectors and host cells having those sequences. The library of randomized sequences is useful for screening for improved expression of any protein of interest, including therapeutic proteins, hormones, a growth factors, extracellular receptors or ligands, proteases, kinases, blood proteins, chemokines, cytokines, antibodies and the like.

### CROSS REFERENCE TO RELATED APPLICATION

This application claims the benefit of U.S. Provisional Application Ser. No. 60/953,813, filed Aug. 3, 2007, the contents of which are herein incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named “346537_SequenceListing.txt”, created on Jul. 30, 2008, and having a size of 3 kilobytes and is filed concurrently with the specification. The sequence listing contained in this ASCII formatted document is part of the specification and is herein incorporated by reference in its entirety.

### FIELD OF THE INVENTION

This invention is in the field of protein production, particularly to the use of modified ribosomal binding site sequences for the production of properly processed heterologous proteins.

### BACKGROUND OF THE INVENTION

More than 150 recombinantly produced proteins and polypeptides have been approved by the U.S. Food and Drug Administration (FDA) for use as biotechnology drugs and vaccines, with another 370 in clinical trials. Unlike small molecule therapeutics that are produced through chemical synthesis, proteins and polypeptides are most efficiently produced in living cells. However, current methods of production of recombinant proteins in bacteria often produce improperly folded, aggregated or inactive proteins, and many types of proteins require secondary modifications that are inefficiently achieved using known methods.

Numerous attempts have been developed to increase production of proteins in recombinant systems. The level of production of a protein in a host cell is determined by several factors, including, for example, the number of copies of its structural gene within a cell and the transcription and translation efficiency. The transcription and translation efficiencies are, in turn, dependent on nucleotide sequences that are normally situated ahead of the desired structural genes or the translated sequence. In most prokaryotes, the purine-rich ribosome site known as the Shine-Dalgarno sequence (or ribosomal binding site, RBS) assists with the binding and positioning of the 30S ribosome component relative to the start codon of the mRNA through interaction with a pyrimidine-rich region of the 16S ribosomal RNA (Shine and Dalgarno (1976) Proc. Natl. Acad. Sci. USA 71: 1342-1346). Prior attempts have been made to increase the efficiency of ribosomal binding, positioning, and translation, by changing the distance between the RBS sequence and the start codon, changing the composition of the space between the RBS sequence and the start codon, modifying an existing RBS sequence to increase the translational efficiency, using a heterologous RBS sequence, and manipulating the secondary structure of mRNA during initiation of translation (Bottaro et al. (1989) DNA 8(5):369-375; PCT Application Publication No. WO 2001098453; Mattanonich et al. (1996) Annals of the New York Academy of Sciences 782:182-190; Weyens et al. (1988) Journal of Molecular Biology 204(4):1045-1048).

### SUMMARY OF THE INVENTION

The present invention provides improved compositions and methods for producing high levels of properly processed protein or polypeptide of interest in a cell expression system. In particular, the invention provides a library of randomized RBS sequences for optimizing heterologous expression of a polypeptide of interest in a host cell. The protein produced by the methods described herein exhibits one or more of improved expression, improved activity, improved solubility, or improved translocation compared to a protein expressed from a polynucleotide comprising a canonical RBS sequence.

Expression constructs comprising the randomized RBS sequences are useful in host cells to express recombinant proteins. Host cells include eukaryotic cells, including yeast cells, insect cells, mammalian cells, plant cells, etc., and prokaryotic cells, including bacterial cells such as P. fluorescens, E. coli, and the like.

As indicated the library of randomized RBS sequences may be used to identify an optimal RBS sequence for expression of a heterologous protein in properly processed form. Any protein of interest may be expressed using the RBS sequences of the invention, including therapeutic proteins, hormones, a growth factors, extracellular receptors or ligands, proteases, kinases, blood proteins, chemokines, cytokines, antibodies and the like.

### BRIEF DESCRIPTION OF THE FIGURES

FIG. 1 depicts the creation of a unique BspEI restriction site within the COP-GFP coding sequence (SEQ ID NO:9). A single base pair mutation was introduced by PCR amplification to create the silent codon mutation: TCC to TCG (serine).

FIG. 2 shows the RC-RBS oligonucleotide (SEQ ID NO: 10) used to construct the RBS library. The RC-RBS oligonucleotide and fill-in primer RC-348 were used to generate the randomized ribosome-binding site (RBS) library fragment.

FIGS. 3A and 3B represent growth plots from the initial assessment of RBS isolates (A and B).

FIGS. 4A and 4B represent a plot of culture broth fluorescence measurements from initial assessment of RBS isolates.

FIG. 5 represents the growth plot for the second assessment of select RBS isolates.

FIG. 6 is a plot of culture broth fluorescence measurements for the second assessment of select RBS isolates.

### DETAILED DESCRIPTION

### Overview

Heterologous protein production often leads to the formation of insoluble or improperly folded proteins, which are difficult to recover and may be inactive. Extremely high expression levels can prevent full translational modifications of the protein to occur, resulting in aggregation and accumulation of uncleaved precursor protein. Modulating translation strength by altering the translation initiation region of a protein of interest can be used to improve the production of heterologous cytoplasmic proteins that accumulate mainly as inclusion bodies due to a translation rate that is too rapid. Secretion of heterologous proteins into the periplasmic space of bacterial cells can also be enhanced by optimizing rather than maximizing protein translation levels such that the translation rate is in sync with the protein secretion rate.

The translation initiation region has been defined as the sequence extending immediately upstream of the ribosomal binding site (RBS) to approximately 20 nucleotides downstream of the initiation codon (McCarthy et al. (1990) Trends in Genetics 6:78-85, herein incorporated by reference in its entirety). In prokaryotes, alternative RBS sequences can be utilized to optimize translation levels of heterologous proteins by providing translation rates that are decreased with respect to the translation levels using the canonical, or consensus, RBS sequence (AGGAGG; SEQ ID NO: 1) described by Shine and Dalgarno ((1974) Proc. Natl. Acad. Sci. USA 71:1342-1346). By “translation rate” or “translation efficiency” is intended the rate of mRNA translation into proteins within cells. In most prokaryotes, the Shine-Dalgarno sequence assists with the binding and positioning of the 30S ribosome component relative to the start codon on the mRNA through interaction with a pyrimidine-rich region of the 16S ribosomal RNA. The RBS (also referred to herein as the Shine-Dalgarno sequence) is located on the mRNA downstream from the start of transcription and upstream from the start of translation, typically from 4 to 14 nucleotides upstream of the start codon, and more typically from 8 to 10 nucleotides upstream of the start codon. Because of the role of the RBS sequence in translation, there is a direct relationship between the efficiency of translation and the efficiency (or strength) of the RBS sequence.

Thus, provided herein are compositions and methods for identifying an optimal RBS sequence for producing high levels of properly processed heterologous polypeptides in a host cell. In particular, a library of expression constructs is provided, wherein each construct in the library comprises a distinct ribosomal binding site (RBS) sequence. In some embodiments, the distinct RBS sequence comprises SEQ ID NO:2, 3, 4, 5, 6, 7, or 8. An “optimal construct” can be identified or selected based on the quantity, quality, and/or location of the expressed protein of interest compared to the expressed protein of interest using other constructs in the library.

### Compositions

A. Oligonucleotide Libraries

The invention encompasses a library of oligonucleotides comprising novel RBS sequence fragments useful for the heterologous expression of a protein or polypeptide of interest in a bacterial host cell. “Heterologous,” “heterologously expressed,” or “recombinant” generally refers to a gene or protein that is not endogenous to the host cell or is not endogenous to the location in the native genome in which it is present, and has been added to the cell by infection, transfection, microinjection, electroporation, microprojection, or the like. In one embodiment, the library comprises a plurality of oligonucleotides comprising an RBS sequence fragment wherein one or more nucleotides corresponding to the canonical RBS sequence (SEQ ID NO: 1) has been fully randomized. In another embodiment, the library comprises a plurality of oligonucleotides comprising an RBS sequence fragment wherein only the nucleotide positions corresponding to the “core” RBS sequence have been fully randomized, or wherein only 1, 2, 3, 4, or 5 nucleotide positions corresponding to the canonical RBS sequence have been fully randomized. The “core” RBS sequence refers to the nucleotide positions corresponding to nucleotides 1 through 4 of SEQ ID NO: 1 (AGGA). In yet another embodiment, the invention encompasses an isolated oligonucleotide comprising SEQ ID NO:2, 3, 4, 5, 6, 7, or 8. The oligonucleotide sequences are useful for optimizing expression of a heterologous protein in a host cell where the translation efficiency is decreased when compared to the translation efficiency of the protein encoded by a gene comprising the canonical RBS sequence.

B. Expression Vectors

The present invention further encompasses a library of expression vectors wherein each vector comprises one of a plurality of randomized RBS sequence fragments useful for the optimal expression of a heterologous protein of interest. In one embodiment, the vector comprises one of a plurality of oligonucleotides comprising an RBS sequence fragment wherein one or more nucleotides corresponding to the canonical RBS sequence (SEQ ID NO: 1) has been fully randomized. In another embodiment, the vector comprises one of a plurality of randomized RBS sequence fragments wherein only the nucleotide positions corresponding to the core RBS sequence have been fully randomized, or wherein only 1, 2, 3, 4, or 5 nucleotide positions corresponding to the canonical RBS sequence have been fully randomized. In yet another embodiment, the vector comprises an RBS sequence fragment wherein the canonical RBS sequence has been replaced by the nucleotide sequence set forth in SEQ ID NO:2, 3, 4, 5, 6, 7, or 8. The library of expression vectors is useful for screening for optimal production of a heterologous protein or polypeptide of interest.

In one embodiment, the vector comprises a polynucleotide sequence of interest operably linked to a promoter. Expressible coding sequences will be operatively attached to a transcription promoter capable of functioning in the chosen host cell, as well as all other required transcription and translation regulatory elements. The coding sequence can be a native coding sequence for the polypeptide of interest, or it can be a coding sequence that has been selected, improved, or optimized for use in the selected expression host cell: for example, by synthesizing the gene to reflect the codon use bias of a host species. The term “operably linked” refers to any configuration in which the transcriptional and any translational regulatory elements are covalently attached to the encoding sequence in such disposition(s), relative to the coding sequence, that in and by action of the host cell, the regulatory elements can direct the expression of the coding sequence.

The vector will typically comprise one or more phenotypic selectable markers and an origin of replication to ensure maintenance of the vector and, if desired, to provide amplification within the host. In one embodiment, the vector further comprises a coding sequence for expression of a protein or polypeptide of interest, operably linked to a leader or secretion signal sequence. The recombinant proteins and polypeptides can be expressed from polynucleotides in which the polypeptide coding sequence is operably linked to the leader sequence and transcription and translation regulatory elements to form a functional gene from which the host cell can express the protein or polypeptide.

Gram-negative bacteria have evolved numerous systems for the active export of proteins across their dual membranes. These routes of secretion include, e.g.: the ABC (Type I) pathway, the Path/Fla (Type III) pathway, and the Path % Vir (Type IV) pathway for one-step translocation across both the plasma and outer membrane; the Sec (Type II), Tat, MscL, and Holins pathways for translocation across the plasma membrane; and the Sec-plus-fimbrial usher porin (FUP), Sec-plus-autotransporter (AT), Sec-plus-two partner secretion (TPS), Sec-plus-main terminal branch (MTB), and Tat-plus-MTB pathways for two-step translocation across the plasma and outer membranes. In one embodiment, the signal sequences useful in the methods of the invention comprise the Sec secretion system signal sequences. (see, Agarraberes and Dice (2001) Biochim Biophys Acta. 1513:1-24; Muller et al. (2001) Prog Nucleic Acid Res Mol. Biol. 66:107-157; U.S. Patent Application Nos. 60/887,476 and 60/887,486, filed Jan. 31, 2007, each of which is herein incorporated by reference in its entirety).

Other regulatory elements may be included in a vector (also termed “expression construct”). Such elements include, but are not limited to, for example, transcriptional enhancer sequences, translational enhancer sequences, other promoters, activators, translational start and stop signals, transcription terminators, cistronic regulators, polycistronic regulators, tag sequences, such as nucleotide sequence “tags” and “tag” polypeptide coding sequences, which facilitates identification, separation, purification, and/or isolation of an expressed polypeptide.

In another embodiment, the expression vector further comprises a tag sequence adjacent to the coding sequence for the protein or polypeptide of interest (or adjacent to the leader or signal sequence if applicable). In one embodiment, this tag sequence allows for purification of the protein. The tag sequence can be an affinity tag, such as a hexa-histidine affinity tag. In another embodiment, the affinity tag can be a glutathione-S-transferase molecule. The tag can also be a fluorescent molecule, such as yellow-fluorescent protein (YFP) or green-fluorescent protein (GFP), or analogs of such fluorescent proteins. The tag can also be a portion of an antibody molecule, or a known antigen or ligand for a known binding partner useful for purification.

A protein-encoding gene according to the present invention can include, in addition to the protein coding sequence comprising the alternate RBS sequence fragment, the following regulatory elements operably linked thereto: a promoter, a transcription terminator, and translational start and stop signals. Examples of methods, vectors, and translation and transcription elements, and other elements useful in the present invention are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox, each of which is herein incorporated by reference in its entirety.

Generally, the recombinant expression vectors will include origins of replication and selectable markers permitting transformation of the host cell and a promoter to direct transcription of the gene of interest. Such promoters can be derived from operons encoding the enzymes such as 3-phosphoglycerate kinase (PGK), acid phosphatase, or heat shock proteins, among others. The gene of interest is assembled in appropriate phase with regulatory sequences as well as translation initiation and termination sequences. Optionally the heterologous sequence can encode a fusion protein including an N-terminal identification polypeptide imparting desired characteristics, e.g., stabilization or simplified purification of expressed recombinant product, as discussed elsewhere herein.

Vectors are known in the art for expressing recombinant proteins in host cells, and any of these may be used for expressing the genes according to the present invention. Such vectors include, e.g., plasmids, cosmids, and phage expression vectors. Examples of useful plasmid vectors include, but are not limited to, the expression plasmids pBBR1MCS, pDSK519, pKT240, pML122, pPS10, RK2, RK6, pRO1600, and RSF1010. Other examples of such useful vectors include those described by, e.g.: N. Hayase, in Appl. Envir. Microbiol. 60(9):3336-42 (September 1994); A. A. Lushnikov et al., in Basic Life Sci. 30: 657-62 (1985); S. Graupner & W. Wackemagel, in Biomolec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96: 47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23: 69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001).

Further examples of expression vectors that can be useful in a host cell comprising the gene of interest comprising one of the randomized RBS sequence fragments of the invention include those listed in Table 1 as derived from the indicated replicons.

TABLE 1
Examples of Useful Expression Vectors

| Replicon | Vector(s) |
|---|---|
| PPS10 | PCN39, PCN51 |
| RSF1010 | PKT261-3 |
| | PMMB66EH |
| | PEB8 |
| | PPLGN1 |
| | PMYC1050 |
| RK2/RP1 | PRK415 |
| | PJB653 |
| PRO1600 | PUCP |
| | PBSP |

The expression plasmid, RSF1010, is described, e.g., by F. Heffron et al., in Proc. Nat'l Acad. Sci. USA 72(9):3623-27 (September 1975), and by K. Nagahari & K. Sakaguchi, in J. Bact. 133(3):1527-29 (March 1978). Plasmid RSF110 and derivatives thereof are particularly useful vectors in the present invention. Exemplary, useful derivatives of RSF1010, which are known in the art, include, e.g., pKT212, pKT214, pKT231 and related plasmids, and pMYC1050 and related plasmids (see, e.g., U.S. Pat. Nos. 5,527,883 and 5,840,554 to Thompson et al.), such as, e.g., pMYC1803. Plasmid pMYC1803 is derived from the RSF1010-based plasmid pTJS260 (see U.S. Pat. No. 5,169,760 to Wilcox), which carries a regulated tetracycline resistance marker and the replication and mobilization loci from the RSF 1010 plasmid. Other exemplary useful vectors include those described in U.S. Pat. No. 4,680,264 to Puhler et al.

In one embodiment, an expression plasmid is used as the expression vector. In another embodiment, RSF 1010 or a derivative thereof is used as the expression vector. In still another embodiment, pMYC1050 or a derivative thereof, or pMYC4803 or a derivative thereof, is used as the expression vector.

The plasmid can be maintained in the host cell by inclusion of a selection marker gene in the plasmid. This may be an antibiotic resistance gene(s), where the corresponding antibiotic(s) is added to the fermentation medium, or any other type of selection marker gene known in the art, e.g., a prototrophy-restoring gene where the plasmid is used in a host cell that is auxotrophic for the corresponding trait, e.g., a biocatalytic trait such as an amino acid biosynthesis or a nucleotide biosynthesis trait, or a carbon source utilization trait.

The promoters used in accordance with the present invention may be constitutive promoters or regulated promoters. Common examples of useful regulated promoters include those of the family derived from the lac promoter (i.e. the lacZ promoter), especially the tac and trc promoters described in U.S. Pat. No. 4,551,433 to DeBoer, as well as Ptac16, Ptac17, PtacII, PlacUV5, and the T7lac promoter. In one embodiment, the promoter is not derived from the host cell organism. In certain embodiments, the promoter is derived from an E. coli organism.

Common examples of non-lac-type promoters useful in expression systems according to the present invention include, e.g., those listed in Table 2.

TABLE 2
Examples of non-lac Promoters

| Promoter | Inducer |
|---|---|
| P_{R} | High temperature |
| P_{L} | High temperature |
| Pm | Alkyl- or halo-benzoates |
| Pu | Alkyl- or halo-toluenes |
| Psal | Salicylates |

See, e.g.: J. Sanchez-Romero & V. De Lorenzo (1999) Genetic Engineering of Nonpathogenic Pseudomonas strains as Biocatalysts for Industrial and Environmental Processes, in Manual of Industrial Microbiology and Biotechnology (A. Demain & J. Davies, eds.) pp. 460-74 (ASM Press, Washington, D.C.); H. Schweizer (2001) Vectors to express foreign genes and techniques to monitor gene expression for Pseudomonads, Current Opinion in Biotechnology, 12: 439-445; and R. Slater & R. Williams (2000) The Expression of Foreign DNA in Bacteria, in Molecular Biology and Biotechnology (J. Walker & R. Rapley, eds.) pp. 125-54 (The Royal Society of Chemistry, Cambridge, UK)). A promoter having the nucleotide sequence of a promoter native to the selected bacterial host cell may also be used to control expression of the gene of interest, e.g., a Pseudomonas anthranilate or benzoate operon promoter (Pant, Pben). Tandem promoters may also be used in which more than one promoter is covalently attached to another, whether the same or different in sequence, e.g., a Pant-Pben tandem promoter (interpromoter hybrid) or a Plac-Plac tandem promoter, or whether derived from the same or different organisms.

Regulated promoters utilize promoter regulatory proteins in order to control transcription of the gene of which the promoter is a part. Where a regulated promoter is used herein, a corresponding promoter regulatory protein will also be part of an expression system according to the present invention. Examples of promoter regulatory proteins include: activator proteins, e.g., E. coli catabolite activator protein, MalT protein; AraC family transcriptional activators; repressor proteins, e.g., E. coli LacI proteins; and dual-function regulatory proteins, e.g., E. coli NagC protein. Many regulated-promoter/promoter-regulatory-protein pairs are known in the art.

Promoter regulatory proteins interact with an effector compound, i.e. a compound that reversibly or irreversibly associates with the regulatory protein so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter/promoter-regulatory-protein/effector-compound trios are known in the art. Although an effector compound can be used throughout the cell culture or fermentation, in a preferred embodiment in which a regulated promoter is used, after growth of a desired quantity or density of host cell biomass, an appropriate effector compound is added to the culture to directly or indirectly result in expression of the desired gene(s) encoding the protein or polypeptide of interest.

By way of example, where a lac family promoter is utilized, a lacI gene can also be present in the system. The lacI gene, which is (normally) a constitutively expressed gene, encodes the Lac repressor protein (LacD protein) which binds to the lac operator of these promoters. Thus, where a lac family promoter is utilized, the lacI gene can also be included and expressed in the expression system. In the case of the lac promoter family members, e.g., the tac promoter, the effector compound is an inducer, preferably a gratuitous inducer such as IPTG (isopropyl-D-1-thiogalactopyranoside, also called “isopropylthiogalactoside”).

For expression of a protein or polypeptide of interest, any plant promoter may also be used. A promoter may be a plant RNA polymerase II promoter. Elements included in plant promoters can be a TATA box or Goldberg-Hogness box, typically positioned approximately 25 to 35 basepairs upstream (5′) of the transcription initiation site, and the CCAAT box, located between 70 and 100 basepairs upstream. In plants, the CCAAT box may have a different consensus sequence than the functionally analogous sequence of mammalian promoters (Messing et al. (1983) In: Genetic Engineering of Plants, Kosuge et al., eds., pp. 211-227). In addition, virtually all promoters include additional upstream activating sequences or enhancers (Benoist and Chambon (1981) Nature 290:304-310; Gruss et al. (1981) Proc. Nat. Acad. Sci. 78:943-947; and Khoury and Gruss (1983) Cell 27:313-314) extending from around −100 bp to −1,000 bp or more upstream of the transcription initiation site.

C. Expression Systems

The present invention provides an improved expression system useful for optimizing production of a heterologous protein or polypeptide of interest. In one embodiment, the system includes a library of expression vectors comprising the gene of interest, wherein the sequence corresponding to the canonical RBS sequence (SEQ ID NO: 1) has been randomized at 1, 2, 3, 4, 5, or all 6 nucleotide positions.

In addition to altering the RBS sequence for optimizing expression, several additional approaches are also encompassed that can be used to control protein translation levels. For example, using promoters with a range of translation strengths, modulating promoter activity by titrating induction, using plasmids with different copy numbers, improving transcript stability, and manipulating sequences other than the RBS sequence in the translation initiation region (see, for example, Simmons and Yansura (1996) Nature Biotechnology 14:629-634, herein incorporated by reference in its entirety).

A particular expression system useful in the methods of the invention includes the Pseudomonads system. The Pseudomonads system offers advantages for commercial expression of polypeptides and enzymes, in comparison with other bacterial expression systems. In particular, P. fluorescens has been identified as an advantageous expression system. P. fluorescens encompasses a group of common, nonpathogenic saprophytes that colonize soil, water and plant surface environments. Commercial enzymes derived from P. fluorescens have been used to reduce environmental contamination, as detergent additives, and for stereoselective hydrolysis. P. fluorescens is also used agriculturally to control pathogens. U.S. Pat. No. 4,695,462 describes the expression of recombinant bacterial proteins in P. fluorescens. Between 1985 and 2004, many companies capitalized on the agricultural use of P. fluorescens for the production of pesticidal, insecticidal, and nematocidal toxins, as well as on specific toxic sequences and genetic manipulation to enhance expression of these. See, for example, PCT Application Nos. WO 03/068926 and WO 03/068948; PCT publication No. WO 03/089455; PCT Application No. WO 04/005221; and, U.S. Patent Publication Number 20060008877.

The pBAD expression system allows tightly controlled, titratable expression of protein or polypeptide of interest through the presence of specific carbon sources such as glucose, glycerol and arabinose (Guzman, et al. (1995) J Bacteriology 177(14): 4121-30). The pBAD vectors are uniquely designed to give precise control over expression levels. Heterologous gene expression from the pBAD vectors is initiated at the araBAD promoter. The promoter is both positively and negatively regulated by the product of the araC gene. AraC is a transcriptional regulator that forms a complex with L-arabinose. In the absence of L-arabinose, the AraC dimer blocks transcription. For maximum transcriptional activation two events are required: (i.) L-arabinose binds to AraC allowing transcription to begin. (ii.) The cAMP activator protein (CAP)-cAMP complex binds to the DNA and stimulates binding of AraC to the correct location of the promoter region.

The trc expression system allows high-level, regulated expression in E. coli from the trc promoter. The trc expression vectors have been optimized for expression of eukaryotic genes in E. coli. The trc promoter is a strong hybrid promoter derived from the tryptophane (trp) and lactose (lac) promoters. It is regulated by the lacO operator and the product of the lacIQ gene (Brosius, J. (1984) Gene 27(2): 161-72).

D. Host Cell

In one embodiment, the host cell useful for the heterologous production of a protein or a polypeptide of interest can be selected from “Gram-negative Proteobacteria Subgroup 18.” “Gram-negative Proteobacteria Subgroup 18” is defined as the group of all subspecies, varieties, strains, and other sub-special units of the species Pseudomonas fluorescens, including those belonging, e.g., to the following (with the ATCC or other deposit numbers of exemplary strain(s) shown in parenthesis): Pseudomonas fluorescens biotype A, also called biovar 1 or biovar I (ATCC 13525); Pseudomonas fluorescens biotype B, also called biovar 2 or biovar II (ATCC 17816); Pseudomonas fluorescens biotype C, also called biovar 3 or biovar III (ATCC 17400); Pseudomonas fluorescens biotype F, also called biovar 4 or biovar IV (ATCC 12983); Pseudomonas fluorescens biotype G, also called biovar 5 or biovar V (ATCC 17518); Pseudomonas fluorescens biovar VI; Pseudomonas fluorescens Pf0-1; Pseudomonas fluorescens Pf-5 (ATCC BAA-477); Pseudomonas fluorescens SBW25; and Pseudomonas fluorescens subsp. cellulosa (NCIMB 10462).

The host cell can be selected from “Gram-negative Proteobacteria Subgroup 19.” “Gram-negative Proteobacteria Subgroup 19” is defined as the group of all strains of Pseudomonas fluorescens biotype A. A particularly preferred strain of this biotype is P. fluorescens strain MB101 (see U.S. Pat. No. 5,169,760 to Wilcox), and derivatives thereof. An example of a preferred derivative thereof is P. fluorescens strain MB214, constructed by inserting into the MB 101 chromosomal asd (aspartate dehydrogenase gene) locus, a native E. coli PlacI-lacI-lacZYA construct (i.e. in which PlacZ was deleted).

Additional P. fluorescens strains that can be used in the present invention include Pseudomonas fluorescens Migula and Pseudomonas fluorescens Loitokitok, having the following ATCC designations: [NCIB 8286]; NRRL B-1244; NCIB 8865 strain CO1; NCIB 8866 strain CO₂; 1291 [ATCC 17458; IFO 15837; NCIB 8917; LA; NRRL B-1864; pyrrolidine; PW2 [ICMP 3966; NCPPB 967; NRRL B-899]; 13475; NCTC 10038; NRRL B-1603 [6; IFO 15840]; 52-1C; CCEB 488-A [BU 140]; CCEB 553 [EM 15/47]; IAM 1008 [AHH-27]; IAM 1055 [AHH-23]; 1 [IFO 15842]; 12 [ATCC 25323; NIH 11; den Dooren de Jong 216]; 18 [IFO 15833; WRRL P-7]; 93 [TR-10]; 108 [52-22; IFO 15832]; 143 [IFO 15836; PL]; 149 [2-40-40; IFO 15838]; 182 [IFO 3081; PJ 73]; 184 [IFO 15830]; 185 [W2 L-1]; 186 [IFO 15829; PJ 79]; 187 [NCPPB 263]; 188 [NCPPB 316]; 189 [PJ227; 1208]; 191 [IFO 15834; PJ 236; 22/1]; 194 [Klinge R-60; PJ 253]; 196 [PJ 288]; 197 [PJ 290]; 198 [PJ 302]; 201 [PJ 368]; 202 [PJ 372]; 203 [PJ 376]; 204 [IFO 15835; PJ 682]; 205 [PJ 686]; 206 [PJ 692]; 207 [PJ 693]; 208 [PJ 722]; 212. [PJ 832]; 215 [PJ 849]; 216 [PJ 885]; 267 [B-9]; 271 [B-1612]; 401 [C71A; IFO 15831; PJ 187]; NRRL B-3178 [4; IFO. 15841]; KY 8521; 3081; 30-21; [IFO 3081]; N; PYR; PW; D946-B83 [BU 2183; FERM-P 3328]; P-2563 [FERM-P 2894; IFO 13658]; IAM-1126 [43F]; M-1; A506 [A5-06]; A505 [A5-05-1]; A526 [A5-26]; B69; 72; NRRL B-4290; PMW6 [NCIB 11615]; SC 12936; Al [IFO 15839]; F 1847 [CDC-EB]; F 1848 [CDC 93]; NCIB 10586; P17; F-12; AmMS 257; PRA25; 6133D02; 6519E01; Ni; SC15208; BNL-WVC; NCTC 2583 [NCIB 8194]; H13; 1013 [ATCC 11251; CCEB 295]; IFO 3903; 1062; or Pf-5.

In one embodiment, the host cell can be any cell capable of producing a protein or polypeptide of interest, including a P. fluorescens cell as described above. The most commonly used systems to produce proteins or polypeptides of interest include certain bacterial cells, particularly E. coli, because of their relatively inexpensive growth requirements and potential capacity to produce protein in large batch cultures. Yeasts are also used to express biologically relevant proteins and polypeptides, particularly for research purposes. Systems include Saccharomyces cerevisiae or Pichia pastoris. These systems are well characterized, provide generally acceptable levels of total protein expression and are comparatively fast and inexpensive. Insect cell expression systems have also emerged as an alternative for expressing recombinant proteins in biologically active form. In some cases, correctly folded proteins that are post-translationally modified can be produced. Mammalian cell expression systems, such as Chinese hamster ovary cells, have also been used for the expression of proteins or polypeptides of interest. On a small scale, these expression systems are often effective. Certain biologics can be derived from proteins, particularly in animal or human health applications. In another embodiment, the host cell is a plant cell, including, but not limited to, a tobacco cell, corn, a cell from an Arabidopsis species, potato or rice cell. In another embodiment, a multicellular organism is analyzed or is modified in the process, including but not limited to a transgenic organism. Techniques for analyzing and/or modifying a multicellular organism are generally based on techniques described for modifying cells described below.

In another embodiment, the host cell can be a prokaryote such as a bacterial cell including, but not limited to an Escherichia or a Pseudomonas species. Typical bacterial cells are described, for example, in “Biological Diversity: Bacteria and Archaeans”, a chapter of the On-Line Biology Book, provided by Dr M J Farabee of the Estrella Mountain Community College, Arizona, USA at the website www.emc.maricotpa.edu/faculty/farabee/BIOBK/BioBookDiversity. In certain embodiments, the host cell can be a Pseudomonad cell, and can typically be a P. fluorescens cell. In other embodiments, the host cell can also be an E. coli cell. In another embodiment the host cell can be a eukaryotic cell, for example an insect cell, including but not limited to a cell from a Spodoptera, Trichoplusia, Drosophila or an Estigmene species, or a mammalian cell, including but not limited to a murine cell, a hamster cell, a monkey, a primate or a human cell.

In one embodiment, the host cell can be a member of any of the bacterial taxa. The cell can, for example, be a member of any species of eubacteria. The host can be a member of any one of the taxa: Acidobacteria, Actinobacteira, Aquificae, Bacteroidetes, Chlorobi, Chlamydiae, Choroflexi, Chrysiogenetes, Cyanobacteria, Deferribacteres, Deinococcus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermomicrobia, Thermotogae, Thermus (Thermales), or Verrucomicrobia. In a embodiment of a eubacterial host cell, the cell can be a member of any species of eubacteria, excluding Cyanobacteria.

The bacterial host can also be a member of any species of Proteobacteria. A proteobacterial host cell can be a member of any one of the taxa Alphaproteobacteria, Betaproteobacteria, Gammaproteobacteria, Deltaproteobacteria, or Epsilonproteobacteria. In addition, the host can be a member of any one of the taxa Alphaproteobacteria, Betaproteobacteria, or Gammaproteobacteria, and a member of any species of Gammaproteobacteria.

In one embodiment of a Gamma Proteobacterial host, the host will be a member of any one of the taxa Aeromonadales, Alteromonadales, Enterobacteriales, Pseudomonadales, or Xanthomonadales; or a member of any species of the Enterobacteriales or Pseudomonadales. In one embodiment, the host cell can be of the order Enterobacteriales, the host cell will be a member of the family Enterobacteriaceae, or may be a member of any one of the genera Erwinia, Escherichia, or Serratia; or a member of the genus Escherichia. Where the host cell is of the order Pseudomonadales, the host cell may be a member of the family Pseudomonadaceae, including the genus Pseudomonas. Gamma Proteobacterial hosts include members of the species Escherichia coli and members of the species Pseudomonas fluorescens.

Other Pseudomonas organisms may also be useful. Pseudomonads and closely related species include Gram-negative Proteobacteria Subgroup 1, which include the group of Proteobacteria belonging to the families and/or genera described as “Gram-Negative Aerobic Rods and Cocci” by R. E. Buchanan and N. E. Gibbons (eds.), Bergey's Manual of Determinative Bacteriology, pp. 217-289 (8th ed., 1974) (The Williams & Wilkins Co., Baltimore, Md., USA) (hereinafter “Bergey (1974)”). Table 3 presents these families and genera of organisms.

TABLE 3
Families and Genera Listed in the Part, “Gram-Negative
Aerobic Rods and Cocci” (in Bergey (1974))

| | |
|---|---|
| Family I. Pseudomomonaceae | Gluconobacter |
| | Pseudomonas |
| | Xanthomonas |
| | Zoogloea |
| Family II. Azotobacteraceae | Azomonas |
| | Azotobacter |
| | Beijerinckia |
| | Derxia |
| Family III. Rhizobiaceae | Agrobacterium |
| | Rhizobium |
| Family IV. Methylomonadaceae | Methylococcus |
| | Methylomonas |
| Family V. Halobacteriaceae | Halobacterium |
| | Halococcus |
| Other Genera | Acetobacter |
| | Alcaligenes |
| | Bordetella |
| | Brucella |
| | Francisella |
| | Thermus |

“Gram-negative Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey (1974). In addition hosts can include cells from the genus Pseudomonas, Pseudomonas enalia (ATCC 14393), Pseudomonas nigrifaciensi (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida. “Gram-negative Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera otherwise described herein, further Proteobacterial genera falling within “Gram-negative Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus Liberibacter”), and Sinorhizobium; and 4) Methylococcaceae family bacteria of the genera Methylobacter, Methylocaldum, Methylomicrobium, Methylosarcina, and Methylosphaera.

In another embodiment, the host cell is selected from “Gram-negative Proteobacteria Subgroup 2.” “Gram-negative Proteobacteria Subgroup 2” is defined as the group of Proteobacteria of the following genera (with the total numbers of catalog-listed, publicly-available, deposited strains thereof indicated in parenthesis, all deposited at ATCC, except as otherwise indicated): Acidomonas (2); Acetobacter (93); Gluconobacter (37); Brevundimonas (23); Beyerinckia (13); Derxia (2); Brucella (4); Agrobacterium (79); Chelatobacter (2); Ensifer (3); Rhizobium (144); Sinorhizobium (24); Blastomonas (1); Sphingomonas (27); Alcaligenes (88); Bordetella (43); Burkholderia (73); Ralstonia (33); Acidovorax (20); Hydrogenophaga (9); Zoogloea (9); Methylobacter (2); Methylocaldum (1 at NCIMB); Methylococcus (2); Methylomicrobium (2); Methylomonas (9); Methylosarcina (1); Methylosphaera; Azomonas (9); Azorhizophilus (5); Azotobacter (64); Cellvibrio (3); Oligella (5); Pseudomonas (1139); Francisella (4); Xanthomonas (229); Stenotrophomonas (50); and Oceanimonas (4).

Exemplary host cell species of “Gram-negative Proteobacteria Subgroup 2” include, but are not limited to the following bacteria (with the ATCC or other deposit numbers of exemplary strain(s) thereof shown in parenthesis): Acidomonas methanolica (ATCC 43581); Acetobacter aceti (ATCC 15973); Gluconobacter oxydans (ATCC 19357); Brevundimonas diminuta (ATCC 11568); Beijerinckia indica (ATCC 9039 and ATCC 19361); Derxia gummosa (ATCC 15994); Brucella melitensis (ATCC 23456), Brucella abortus (ATCC 23448); Agrobacterium tumefaciens (ATCC 23308), Agrobacterium radiobacter (ATCC 19358), Agrobacterium rhizogenes (ATCC 11325); Chelatobacter heintzii (ATCC 29600); Ensifer adhaerens (ATCC 33212); Rhizobium leguminosarum (ATCC 10004); Sinorhizobium fredii (ATCC 35423); Blastomonas natatoria (ATCC 35951); Sphingomonas paucimobilis (ATCC 29837); Alcaligenes faecalis (ATCC 8750); Bordetella pertussis (ATCC 9797); Burkholderia cepacia (ATCC 25416); Ralstonia pickettii (ATCC 27511); Acidovorax facilis (ATCC 11228); Hydrogenophaga flava (ATCC 33667); Zoogloea ramigera (ATCC 19544); Methylobacter luteus (ATCC 49878); Methylocaldum gracile (NCIMB 11912); Methylococcus capsulatus (ATCC 19069); Methylomicrobium agile (ATCC 35068); Methylomonas methanica (ATCC 35067); Methylosarcina fibrata (ATCC 700909); Methylosphaera hansonii (ACAM 549); Azomonas agilis (ATCC 7494); Azorhizophilus paspali (ATCC 23833); Azotobacter chroococcum (ATCC 9043); Cellvibrio mixtus (UQM 2601); Oligella urethralis (ATCC 17960); Pseudomonas aeruginosa (ATCC 10145), Pseudomonas fluorescens (ATCC 35858); Francisella tularensis (ATCC 6223); Stenotrophomonas maltophilia (ATCC 13637); Xanthomonas campestris (ATCC 33913); and Oceanimonas doudoroffli (ATCC 27123).

In another embodiment, the host cell is selected from “Gram-negative Proteobacteria Subgroup 3.” “Gram-negative Proteobacteria Subgroup 3” is defined as the group of Proteobacteria of the following genera: Brevundimonas; Agrobacterium; Rhizobium; Sinorhizobium; Blastomonas; Sphingomonas; Alcaligenes; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In another embodiment, the host cell is selected from “Gram-negative Proteobacteria Subgroup 4.” “Gram-negative Proteobacteria Subgroup 4” is defined as the group of Proteobacteria of the following genera: Brevundimonas; Blastomonas; Sphingomonas; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In another embodiment, the host cell is selected from “Gram-negative Proteobacteria Subgroup 5.” “Gram-negative Proteobacteria Subgroup 5” is defined as the group of Proteobacteria of the following genera: Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

The host cell can be selected from “Gram-negative Proteobacteria Subgroup 6.” “Gram-negative Proteobacteria Subgroup 6” is defined as the group of Proteobacteria of the following genera: Brevundimonas; Blastomonas; Sphingomonas; Burkholderia; Ralstonia; Acidovorax; Hydrogenophaga; Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram-negative Proteobacteria Subgroup 7.” “Gram-negative Proteobacteria Subgroup 7” is defined as the group of Proteobacteria of the following genera: Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram-negative Proteobacteria Subgroup 8.” “Gram-negative Proteobacteria Subgroup 8” is defined as the group of Proteobacteria of the following genera: Brevundimonas; Blastomonas; Sphingomonas; Burkholderia; Ralstonia; Acidovorax; Hydrogenophaga; Pseudomonas; Stenotrophomonas; Xanthomonas; and Oceanimonas.

The host cell can be selected from “Gram-negative Proteobacteria Subgroup 9.” “Gram-negative Proteobacteria Subgroup 9” is defined as the group of Proteobacteria of the following genera: Brevundimonas; Burkholderia; Ralstonia; Acidovorax; Hydrogenophaga; Pseudomonas; Stenotrophomonas; and Oceanimonas.

The host cell can be selected from “Gram-negative Proteobacteria Subgroup 10.” “Gram-negative Proteobacteria Subgroup 10” is defined as the group of Proteobacteria of the following genera: Burkholderia; Ralstonia; Pseudomonas; Stenotrophomonas; and Xanthomonas.

The host cell can be selected from “Gram-negative Proteobacteria Subgroup 11.” “Gram-negative Proteobacteria Subgroup 11” is defined as the group of Proteobacteria of the genera: Pseudomonas; Stenotrophomonas; and Xanthomonas. The host cell can be selected from “Gram-negative Proteobacteria Subgroup 12.” “Gram-negative Proteobacteria Subgroup 12” is defined as the group of Proteobacteria of the following genera: Burkholderia; Ralstonia; Pseudomonas. The host cell can be selected from “Gram-negative Proteobacteria Subgroup 13.” “Gram-negative Proteobacteria Subgroup 13” is defined as the group of Proteobacteria of the following genera: Burkholderia; Ralstonia; Pseudomonas; and Xanthomonas. The host cell can be selected from “Gram-negative Proteobacteria Subgroup 14.” “Gram-negative Proteobacteria Subgroup 14” is defined as the group of Proteobacteria of the following genera: Pseudomonas and Xanthomonas. The host cell can be selected from “Gram-negative Proteobacteria Subgroup 15.” “Gram-negative Proteobacteria Subgroup 15” is defined as the group of Proteobacteria of the genus Pseudomonas.

The host cell can be selected from “Gram-negative Proteobacteria Subgroup 16.” “Gram-negative Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoalcaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas aspleni (ATCC 23835); Pseudomonas azelaica (ATCC 27162); Pseudomonas beyerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonas flectens (ATCC 12775); Pseudomonas azotoformans; Pseudomonas brenneri; Pseudomonas cedrella; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginata (ATCC 25417); Pseudomonas mephitica (ATCC 33665); Pseudomonas denitrificans (ATCC 19244); Pseudomonas pertucinogena (ATCC 190); Pseudomonas pictorum (ATCC 23328); Pseudomonas psychrophila; Pseudomonas filva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis.

The host cell can be selected from “Gram-negative Proteobacteria Subgroup 17.” “Gram-negative Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

Other suitable hosts include those classified in other parts of the reference, such as Gram (+) Proteobacteria. In one embodiment, the host cell is an E. coli. The genome sequence for E. coli has been established for E. coli MG1655 (Blattner, et al. (1997) The complete genome sequence of Escherichia coli K-12, Science 277(5331): 1453-74) and DNA microarrays are available commercially for E. coli K₁₂ (MWG Inc, High Point, N.C.). E. coli can be cultured in either a rich medium such as Luria-Bertani (LB) (10 g/L tryptone, 5 g/L NaCl, 5 g/L yeast extract) or a defined minimal medium such as M9 (6 g/L Na₂HPO₄, 3 g/L KH₂PO₄, 1 g/L NH₄Cl, 0.5 g/L NaCl, pH 7.4) with an appropriate carbon source such as 1% glucose. Routinely, an over night culture of E. coli cells is diluted and inoculated into fresh rich or minimal medium in either a shake flask or a fermentor and grown at 37° C.

A host can also be of mammalian origin, such as a cell derived from a mammal including any human or non-human mammal. Mammals can include, but are not limited to primates, monkeys, porcine, ovine, bovine, rodents, ungulates, pigs, swine, sheep, lambs, goats, cattle, deer, mules, horses, monkeys, apes, dogs, cats, rats, and mice.

A host cell may also be of plant origin. Examples of suitable host cells would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassaya, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radiscchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yams, and zucchini. In some embodiments, plants useful in the method are Arabidopsis, corn, wheat, soybean, and cotton.

E. Kits

The present invention also provides kits useful for identifying an optimal RBS sequence for producing a heterologous protein or polypeptide of interest. The kit comprises a library of oligonucleotides wherein the RBS sequence has been fully randomized. In some embodiments, the library comprises oligonucleotides comprising an RBS sequence that has only been randomized at the core RBS sequence. In another embodiment, the library consists of oligonucleotides comprising SEQ ID NO:2, 3, 4, 5, 6, 7, and 8. The kit may further comprise one or more control oligonucleotides comprising the canonical RBS sequence. These kits may also comprise reagents sufficient for introducing the oligonucleotides into an expression construct comprising a polynucleotide encoding a polypeptide of interest, reagents for introducing the expression construct into a host cell of interest, reagents sufficient to facilitate growth and maintenance of the host cell populations, as well as reagents for expression of the heterologous protein or polypeptide in the host cell. The library may be provided in the kit in any manner suitable for storage, transport, and use of the oligonucleotides.

### Methods

Provided herein are methods for the optimal expression of a gene encoding a polypeptide of interest, wherein the gene comprises an altered RBS sequence. In some embodiments, modification of the RBS sequence results in a decrease in the translation rate of the polypeptide of interest. While not being bound to any particular theory or mechanism, this decrease in translation rate may correspond to an increase in the level of properly processed protein or polypeptide per gram of protein produced, or per gram of host protein. The decreased translation rate can also correlate with an increased level of recoverable protein or polypeptide produced per gram of recombinant or per gram of host cell protein. The decreased translation rate can also correspond to any combination of an increased expression, increased activity, increased solubility, or increased translocation (e.g., to a periplasmic compartment or secreted into the extracellular space). In this embodiment, the term “increased” is relative to the level of protein or polypeptide that is produced, properly processed, soluble, and/or recoverable when the protein or polypeptide of interest is expressed under the same conditions, and wherein the nucleotide sequence encoding the polypeptide comprises the canonical RBS sequence. Similarly, the term “decreased” is relative to the translation rate of the protein or polypeptide of interest wherein the gene encoding the protein or polypeptide comprises the canonical RBS sequence. The translation rate can be decreased by at least about 5%, at least about 10%, at least about 15%, at least about 20%, about 25%, about 30%, about 35%, about 40%, about 45%, about 50%, about 55%, about 60%, about 65%, about 70, at least about 75% or more, or at least about 2-fold, about 3-fold, about 4-fold, about 5-fold, about 6-fold, about 7-fold, or greater.

In some embodiments, the RBS sequence variants described herein can be classified as resulting in high, medium, or low translation efficiency. In one embodiment, the sequences are ranked according to the level of translational activity compared to translational activity of the canonical RBS sequence. A high RBS sequence has about 60% to about 100% of the activity of the canonical sequence. A medium RBS sequence has about 40% to about 60% of the activity of the canonical sequence. A low RBS sequence has less than about 40% of the activity of the canonical sequence. Methods for measuring translation efficiency are described elsewhere herein (see, for example, the Experimental Examples).

A. Oligonucleotide Design

The library of RBS sequences can be generated by fully randomizing each position of the canonical RBS sequence (AGGAGG, SEQ ID NO: 1). A fully randomized RBS sequence is represented by the sequence “N,N,N,N,N,N” (corresponding to nucleotide positions 12 through 17 of SEQ ID NO:9) where “N” can be any one of the nucleotide bases A, T, C or G. As used herein, the term “corresponding to” refers to a nucleotide in a first nucleic acid sequence that aligns with a given nucleotide in a reference nucleic acid sequence when the first nucleic acid and reference nucleic acid sequences are aligned. Thus, there are 4096 possible nucleotide sequences represented by a fully randomized RBS sequence that uses A, T, G and C.

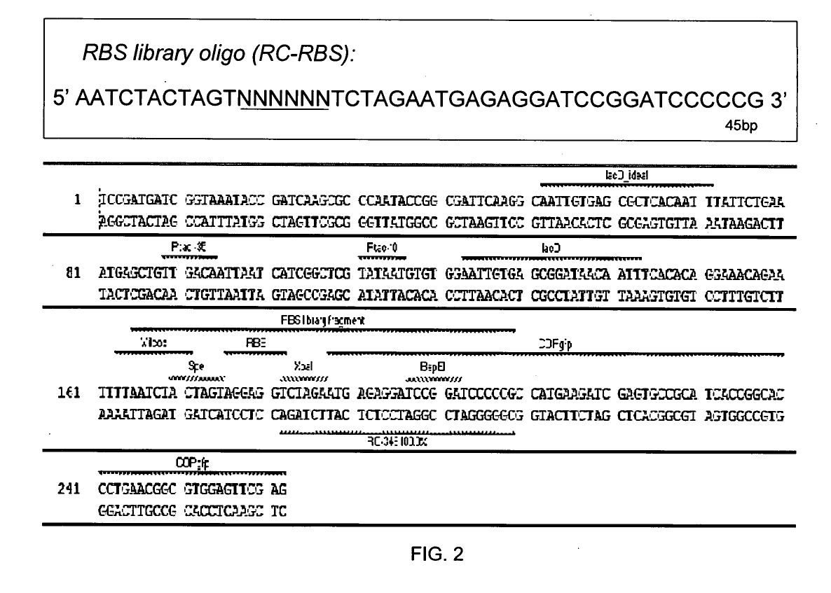

In another embodiment, the RBS is fully randomized only in the “core” sequence, which corresponds to residues 1 through 4 of SEQ ID NO: 1 (AGGA). In yet another embodiment, the RBS is fully randomized in only 1, 2, 3, 4, or 5 of the positions corresponding to SEQ ID NO: 1. The randomized RBS sequence can be generated by using an oligonucleotide corresponding to the translation initiation region of the gene encoding the protein of interest, wherein the oligonucleotide is fully degenerate at one or more positions of the RBS sequence (see FIG. 2).

Oligonucleotides are typically synthesized chemically according to the solid phase phosphoramidite triester method described by Beaucage and Caruthers (1981), Tetrahedron Letts. 22(20):1859-1862, for example, using an automated synthesizer, as described in Needham-VanDevanter et al. (1984) Nucleic Acids Res. 12:6159-6168. A wide variety of equipment is commercially available for automated oligonucleotide synthesis. Multi-nucleotide synthesis approaches (e.g., tri-nucleotide synthesis) are also useful.

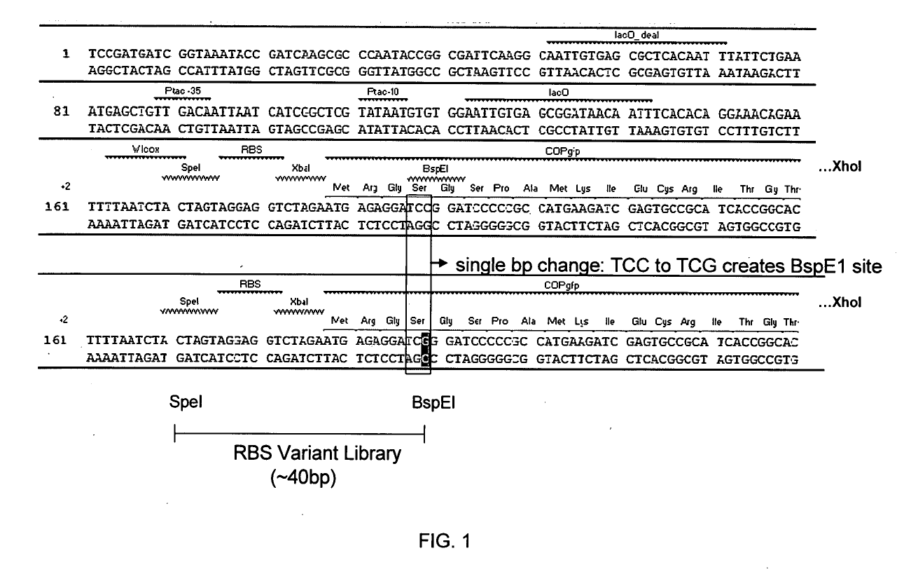

The oligonucleotides are typically designed to incorporate restriction sites to facilitate cloning of the translation initiation region comprising the modified RBS sequences into the expression constructs (see FIG. 1). The restriction sites may occur naturally in the parent nucleotide sequence, or may be inserted into the sequence, for example, using site-directed mutagenesis. Insertion of a restriction site should be done in a manner that does not disrupt the activity or function of the polynucleotide or the encoded polypeptide. Sequences that are cleaved by restriction endonucleases (“restriction sites”) are well known in the art.

B. Library Construction

After designing and synthesizing the population(s) of oligonucleotides encoding the randomized RBS sequences, the oligonucleotides are introduced into the expression construct comprising a polynucleotide encoding the polypeptide of interest. In this context, “introduced” means to insert the sequences of the oligonucleotides comprising the modified RBS into the polynucleotide encoding the polypeptide of interest such that the sequence in the ribosomal binding site region is replaced by the oligonucleotide sequence.

In one embodiment, the population of oligonucleotides is introduced into the expression construct by annealing the oligonucleotides and then ligating the population of oligonucleotides into a vector comprising the polynucleotide encoding the polypeptide of interest to generate a construct library. This can be accomplished, for example, by identifying or introducing (for example, by site-directed mutagenesis) unique restriction sites into the sequences flanking the RBS in the polynucleotide of interest, and designing the oligonucleotide(s) to contain the same unique restriction sites. In this example, the RBS region may be easily replaced by enzymatic digestion with the restriction endonuclease enzyme(s) that will specifically cleave the polynucleotide within the unique restriction site(s) in both the RBS region of the polynucleotide of interest and in the oligonucleotide(s). The digested oligonucleotides are then ligated (e.g., introduced) into the digested vector comprising the polynucleotide of interest using standard molecular biology techniques. The oligonucleotides may be ligated without the need for extension (e.g., polymerase-based chain extension). The resulting library is transformed into a host cell and grown under conditions to facilitate expression of the protein. Methods for assaying function or activity are then utilized to identify the optimal construct for producing the polypeptide of interest.

In another embodiment, the oligonucleotides can be introduced into the polynucleotide of interest using polymerase chain reaction, wherein the oligonucleotides corresponding to the RBS region are annealed to the polynucleotide of interest and the constructs are generated by primer extension using a thermostable DNA polymerase and further techniques well known to those of skill in the art.

Transformation of the host cells with the vector(s) disclosed herein may be performed using any transformation methodology known in the art, and the bacterial host cells may be transformed as intact cells or as protoplasts (i.e. including cytoplasts). Exemplary transformation methodologies include poration methodologies, e.g., electroporation, protoplast fusion, bacterial conjugation, and divalent cation treatment, e.g., calcium chloride treatment or CaCl/Mg2+ treatment, or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

C. Screening for Optimal RBS Sequence

The library of expression constructs described herein can be screened for the optimal RBS sequence for expression of a heterologous protein of interest. The optimal RBS sequence can be identified or selected based on the quantity, quality, and/or location of the expressed protein of interest. In one embodiment, the optimal RBS sequence is one that results in an increased level of total protein, increased level of properly processed protein, or increased level of active or soluble protein within (or secreted from) the host cell compared to other constructs in the library, or to a construct comprising the canonical RBS sequence.

An optimized expression level of a protein or polypeptide of interest can refer to an increase in the solubility of the protein. The protein or polypeptide of interest can be produced and recovered from the cytoplasm, periplasm or extracellular medium of the host cell. The protein or polypeptide can be insoluble or soluble. The protein or polypeptide can include one or more targeting sequences or sequences to assist purification, as discussed supra.

The term “soluble” as used herein means that the protein is not precipitated by centrifugation at between approximately 5,000 and 20,000×gravity when spun for 10-30 minutes in a buffer under physiological conditions. Soluble proteins are not part of an inclusion body or other precipitated mass. Similarly, “insoluble” means that the protein or polypeptide can be precipitated by centrifugation at between 5,000 and 20,000×gravity when spun for 10-30 minutes in a buffer under physiological conditions. Insoluble proteins or polypeptides can be part of an inclusion body or other precipitated mass. The term “inclusion body” is meant to include any intracellular body contained within a cell wherein an aggregate of proteins or polypeptides has been sequestered. In some embodiments, expression of a gene comprising an optimized RBS sequence results in a decrease in the accumulation of insoluble protein in inclusion bodies. The decrease in accumulation may be a decrease of at least about 5%, at least about 10%, at least about 15%, at least about 20%, about 25%, about 30%, about 35%, about 40%, about 45%, about 50%, about 55%, about 60%, about 65%, about 70, at least about 75% or more, or at least about 2-fold, about 3-fold, about 4-fold, about 5-fold, about 6-fold, about 7-fold, or greater.

The methods of the invention can produce protein localized to the periplasm of the host cell. In one embodiment, the optimal RBS sequence results in an increase in the production of properly processed proteins or polypeptides of interest in the cell. In another embodiment, there may be an increase in the production of actve proteins or polypeptides of interest in the cell. The optimal RBS sequence may also lead to an increased yield of active and/or soluble proteins or polypeptides of interest as compared to when the protein is expressed from a gene comprising the canonical RBS sequence.

In one embodiment, the optimal RBS results in the production of at least 0.1 g/L protein in the periplasmic compartment. In another embodiment, the optimal RBS results in the production of 0.1 to 10 g/L periplasmic protein in the cell, or at least about 0.2, about 0.3, about 0.4, about 0.5, about 0.6, about 0.7, about 0.8, about 0.9 or at least about 1.0 g/L periplasmic protein. In one embodiment, the total protein or polypeptide of interest produced is at least 1.0 g/L, at least about 2 g/L, at least about 3 g/L, about 4 g/L, about 5 g/L, about 6 g/L, about 7 g/L, about 8 g/L, about 10 g/L, about 15 g/L, about 20 g/L, at least about 25 g/L, or greater. In some embodiments, the amount of periplasmic protein produced is at least about 5%, about 10%, about 15%, about 20%, about 25%, about 30%, about 40%, about 50%, about 60%, about 70%, about 80%, about 90%, about 95%, about 96%, about 97%, about 98%, about 99%, or more of total protein or polypeptide of interest produced.

In one embodiment, the optimal RBS results in the production of at least 0.1 g/L correctly processed protein. A correctly processed protein has an amino terminus of the native protein. In another embodiment, the optimal RBS results in the production of 0.1 to 10 g/L correctly processed protein in the cell, including at least about 0.2, about 0.3, about 0.4, about 0.5, about 0.6, about 0.7, about 0.8, about 0.9 or at least about 1.0 g/L correctly processed protein. In another embodiment, the total correctly processed protein or polypeptide of interest produced is at least 1.0 g/L, at least about 2 g/L, at least about 3 g/L, about 4 g/L, about 5 g/L, about 6 g/L, about 7 g/L, about 8 g/L, about 10 g/L, about 15 g/L, about 20 g/L, about 25 g/L, about 30 g/L, about 35 g/l, about 40 g/l, about 45 g/l, at least about 50 g/L, or greater. In some embodiments, the amount of correctly processed protein produced is at least about 5%, about 10%, about 15%, about 20%, about 25%, about 30%, about 40%, about 50%, about 60%, about 70%, about 80%, about 90%, about 95%, about 96%, about 97%, about 98%, at least about 99%, or more of total recombinant protein in a correctly processed form.

The optimal RBS can also results in the production of an increased yield of the protein or polypeptide of interest. In one embodiment, the optimal sequences results in the production of a protein or polypeptide of interest as at least about 5%, at least about 10%, about 15%, about 20%, about 25%, about 30%, about 40%, about 45%, about 50%, about 55%, about 60%, about 65%, about 70%, about 75%, or greater of total cell protein (tcp). “Percent total cell protein” is the amount of protein or polypeptide in the host cell as a percentage of aggregate cellular protein. The determination of the percent total cell protein is well known in the art.

In a particular embodiment, the host cell comprising the optimal RBS can have a recombinant polypeptide, polypeptide, protein, or fragment thereof expression level of at least 1% tcp and a cell density of at least 40 g/L, when grown (i.e. within a temperature range of about 4° C. to about 55° C., including about 10° C., about 15° C., about 20° C., about 25° C., about 30° C., about 35° C., about 40° C., about 45° C., and about 50° C.) in a mineral salts medium. In a particularly preferred embodiment, the optimal expression system will have a protein or polypeptide expression level of at least 5% tcp and a cell density of at least 40 g/L, when grown (i.e. within a temperature range of about 4° C. to about 55° C., inclusive) in a mineral salts medium at a fermentation scale of at least about 10 Liters.

In practice, heterologous proteins targeted to the periplasm are often found in the broth (see European Patent No. EP 0 288 451), possibly because of damage to or an increase in the fluidity of the outer cell membrane. The rate of this “passive” secretion may be increased by using a variety of mechanisms that permeabilize the outer cell membrane: colicin (Miksch et al. (1997) Arch. Microbiol. 167: 143-150); growth rate (Shokri et al. (2002) App Miocrobiol Biotechnol 58:386-392); TolIII overexpression (Wan and Baneyx (1998) Protein Expression Purif. 14: 13-22); bacteriocin release protein (Hsiung et al. (1989) Bio/Technology 7: 267-71), colicin A lysis protein (Lloubes et al. (1993) Biochimie 75: 451-8) mutants that leak periplasmic proteins (Furlong and Sundstrom (1989) Developments in Indus. Microbio. 30: 141-8); fusion partners (Jeong and Lee (2002) Appl. Environ. Microbio. 68: 4979-4985); recovery by osmotic shock (Taguchi et al. (1990) Biochimica Biophysica Acta 1049: 278-85). Transport of engineered proteins to the periplasmic space with subsequent localization in the broth has been used to produce properly folded and active proteins in E. coli (Wan and Baneyx (1998) Protein Expression Purif: 14: 13-22; Simmons et al. (2002) J. Immun. Meth. 263: 133-147; Lundell et al. (1990) J. Indust. Microbio. 5: 215-27).

In some embodiments, the methods of the invention result in the identification of an optimal translation initation region sequence that results in an increase in the amount of protein produced in an active form. The term “active” means the presence of biological activity, wherein the biological activity is comparable or substantially corresponds to the biological activity of a corresponding native protein or polypeptide. In the context of proteins this typically means that a polynucleotide or polypeptide comprises a biological function or effect that has at least about 20%, about 50%, preferably at least about 60-80%, and most preferably at least about 90-95% activity compared to the corresponding native protein or polypeptide using standard parameters. The determination of protein or polypeptide activity can be performed utilizing corresponding standard, targeted comparative biological assays for particular proteins or polypeptides. One indication that a protein or polypeptide of interest maintains biological activity is that the polypeptide is immunologically cross reactive with the native polypeptide.

The optimal RBS sequences of the invention can also improve recovery of active protein or polypeptide of interest. Active proteins can have a specific activity of at least about 20%, at least about 30%, at least about 40%, about 50%, about 60%, at least about 70%, about 80%, about 90%, or at least about 95% that of the native protein or polypeptide from which the sequence is derived. Further, the substrate specificity (k_{cat}/Kₘ) is optionally substantially similar to the native protein or polypeptide. Typically, k_{cat}/Kₘ will be at least about 30%, about 40%, about 50%, about 60%, about 70%, about 80%, at least about 90%, at least about 95%, or greater. Methods of assaying and quantifying measures of protein and polypeptide activity and substrate specificity (k_{cat}/Kₘ), are well known to those of skill in the art.

The activity of the protein or polypeptide of interest can be also compared with a previously established native protein or polypeptide standard activity. Alternatively, the activity of the protein or polypeptide of interest can be determined in a simultaneous, or substantially simultaneous, comparative assay with the native protein or polypeptide. For example, in vitro assays can be used to determine any detectable interaction between a protein or polypeptide of interest and a target, e.g. between an expressed enzyme and substrate, between expressed hormone and hormone receptor, between expressed antibody and antigen, etc. Such detection can include the measurement of calorimetric changes, proliferation changes, cell death, cell repelling, changes in radioactivity, changes in solubility, changes in molecular weight as measured by gel electrophoresis and/or gel exclusion methods, phosphorylation abilities, antibody specificity assays such as ELISA assays, etc. In addition, in vivo assays include, but are not limited to, assays to detect physiological effects of the heterologously produced protein or polypeptide in comparison to physiological effects of the native protein or polypeptide, e.g. weight gain, change in electrolyte balance, change in blood clotting time, changes in clot dissolution and the induction of antigenic response. Generally, any in vitro or in vivo assay can be used to determine the active nature of the protein or polypeptide of interest that allows for a comparative analysis to the native protein or polypeptide so long as such activity is assayable. Alternatively, the proteins or polypeptides produced in the present invention can be assayed for the ability to stimulate or inhibit interaction between the protein or polypeptide and a molecule that normally interacts with the protein or polypeptide, e.g. a substrate or a component of the signal pathway that the native protein normally interacts. Such assays can typically include the steps of combining the protein with a substrate molecule under conditions that allow the protein or polypeptide to interact with the target molecule, and detect the biochemical consequence of the interaction with the protein and the target molecule.

Assays that can be utilized to determine protein or polypeptide activity are described, for example, in Ralph, P. J., et al. (1984) J. Immunol. 132:1858 or Saiki et al. (1981) J. Immunol. 127:1044, Steward, W. E. II (1980) The Interferon Systems. Springer-Verlag, Vienna and New York, Broxmeyer, H. E., et al. (1982) Blood 60:595, Molecular Cloning: A Laboratory Manual”, 2d ed., Cold Spring Harbor Laboratory Press, Sambrook, J., E. F. Fritsch and T. Maniatis eds., 1989, and Methods in Enzymology: Guide to Molecular Cloning Techniques, Academic Press, Berger, S. L. and A. R. Kimmel eds., 1987, A K Patra et al., Protein Expr Purif, 18(2): p/182-92 (2000), Kodama et al., J. Biochem. 99: 1465-1472 (1986); Stewart et al., Proc. Natl. Acad. Sci. USA 90: 5209-5213 (1993); (Lombillo et al., J. Cell Biol. 128:107-115 (1995); (Vale et al., Cell 42:39-50 (1985).

D. Cell Growth Conditions

The cell growth conditions for the host cells described herein can include that which facilitates expression of the protein of interest, and/or that which facilitates fermentation of the expressed protein of interest. As used herein, the term “fermentation” includes both embodiments in which literal fermentation is employed and embodiments in which other, non-fermentative culture modes are employed. Fermentation may be performed at any scale. In one embodiment, the fermentation medium may be selected from among rich media, minimal media, and mineral salts media; a rich medium may be used, but is preferably avoided. In another embodiment either a minimal medium or a mineral salts medium is selected. In still another embodiment, a minimal medium is selected. In yet another embodiment, a mineral salts medium is selected. Mineral salts media are particularly preferred.

Mineral salts media consists of mineral salts and a carbon source such as, e.g., glucose, sucrose, or glycerol. Examples of mineral salts media include, e.g., M9 medium, Pseudomonas medium (ATCC 179), Davis and Mingioli medium (see, B D Davis & E S Mingioli (1950) in J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. The mineral salts medium does not have, but can include an organic nitrogen source, such as peptone, tryptone, amino acids, or a yeast extract. An inorganic nitrogen source can also be used and selected from among, e.g., ammonium salts, aqueous ammonia, and gaseous ammonia. In comparison to mineral salts media, minimal media can also contain mineral salts and a carbon source, but can be supplemented with, e.g., low levels of amino acids, vitamins, peptones, or other ingredients, though these are added at very minimal levels.

The expression system according to the present invention can be cultured in any fermentation format. For example, batch, fed-batch, semi-continuous, and continuous fermentation modes may be employed herein. Wherein the protein is excreted into the extracellular medium, continuous fermentation is preferred.

The expression systems according to the present invention are useful for transgene expression at any scale (i.e. volume) of fermentation. Thus, e.g., microliter-scale, centiliter scale, and deciliter scale fermentation volumes may be used; and 1 Liter scale and larger fermentation volumes can be used. In one embodiment, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

In the present invention, growth, culturing, and/or fermentation of the transformed host cells is performed within a temperature range permitting survival of the host cells, preferably a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the host cells of the present invention, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

In some embodiments, the expression system comprises a Pseudomonas host cell, e.g. Psuedomonas fluorescens. An advantage in using Pseudomonas fluorescens in expressing secreted proteins includes the ability of Pseudomonas fluorescens to be grown in high cell densities compared to E. coli or other bacterial expression systems. To this end, Pseudomonas fluorescens expressions systems according to the present invention can provide a cell density of about 20 g/L or more. The Pseudomonas fluorescens expressions systems according to the present invention can likewise provide a cell density of at least about 70 g/L, as stated in terms of biomass per volume, the biomass being measured as dry cell weight.

In one embodiment, the cell density will be at least about 20 g/L. In another embodiment, the cell density will be at least about 25 g/L, about 30 g/L, about 35 g/L, about 40 g/L, about 45 g/L, about 50 g/L, about 60 g/L, about 70 g/L, about 80 g/L, about 90 g/L., about 100 g/L, about 110 g/L, about 120 g/L, about 130 g/L, about 140 g/L, about or at least about 150 g/L.

In another embodiments, the cell density at induction will be between about 20 g/L and about 150 g/L; between about 20 g/L and about 120 g/L; about 20 g/L and about 80 g/L; about 25 g/L and about 80 g/L; about 30 g/L and about 80 g/L; about 35 g/L and about 80 g/L; about 40 g/L and about 80 g/L; about 45 g/L and about 80 g/L; about 50 g/L and about 80 g/L; about 50 g/L and about 75 g/L; about 50 g/L and about 70 g/L; about 40 g/L and about 80 g/L.

E. Isolation of Protein or Polypeptide of Interest

To release targeted proteins from the periplasm, treatments involving chemicals such as chloroform (Ames et al. (1984) J. Bacteriol., 160: 1181-1183), guanidine-HCl, and Triton X-100 (Naglak and Wang (1990) Enzyme Microb. Technol., 12: 603-611) have been used. However, these chemicals are not inert and may have detrimental effects on many recombinant protein products or subsequent purification procedures. Glycine treatment of E. coli cells, causing permeabilization of the outer membrane, has also been reported to release the periplasmic contents (Ariga et al. (1989) J. Ferm. Bioeng., 68: 243-246). The most widely used methods of periplasmic release of recombinant protein are osmotic shock (Nosal and Heppel (1966) J. Biol. Chem., 241: 3055-3062; Neu and Heppel (1965) J. Biol. Chem., 240: 3685-3692), hen eggwhite (HEW)-lysozyme/ethylenediamine tetraacetic acid (EDTA) treatment (Neu and Heppel (1964) J. Biol. Chem., 239: 3893-3900; Witholt et al. (1976) Biochim. Biophys. Acta, 443: 534-544; Pierce et al. (1995) ICheme Research. Event, 2: 995-997), and combined HEW-lysozyme/osmotic shock treatment (French et al. (1996) Enzyme and Microb. Tech., 19: 332-338). The French method involves resuspension of the cells in a fractionation buffer followed by recovery of the periplasmic fraction, where osmotic shock immediately follows lysozyme treatment. The effects of overexpression of the recombinant protein, S. thermoviolaceus α-amylase, and the growth phase of the host organism on the recovery are also discussed.

Typically, these procedures include an initial disruption in osmotically-stabilizing medium followed by selective release in non-stabilizing medium. The composition of these media (pH, protective agent) and the disruption methods used (chloroform, HEW-lysozyme, EDTA, sonication) vary among specific procedures reported. A variation on the HEW-lysozyme/EDTA treatment using a dipolar ionic detergent in place of EDTA is discussed by Stabel et al. (1994) Veterinay Microbiol., 38: 307-314. For a general review of use of intracellular lytic enzyme systems to disrupt E. coli, see Dabora and Cooney (1990) in Advances in Biochemical Engineering/Biotechnology, Vol. 43, A. Fiechter, ed. (Springer-Verlag: Berlin), pp. 11-30.

Conventional methods for the recovery of proteins or polypeptides of interest from the cytoplasm, as soluble protein or refractile particles, involved disintegration of the bacterial cell by mechanical breakage. Mechanical disruption typically involves the generation of local cavitation in a liquid suspension, rapid agitation with rigid beads, sonication, or grinding of cell suspension (Bacterial Cell Surface Techniques, Hancock and Poxton (John Wiley & Sons Ltd, 1988), Chapter 3, p. 55).

HEW-lysozyme acts biochemically to hydrolyze the peptidoglycan backbone of the cell wall. The method was first developed by Zinder and Arndt (1956) Proc. Natl. Acad. Sci. USA, 42: 586-590, who treated E. coli with egg albumin (which contains HEW-lysozyme) to produce rounded cellular spheres later known as spheroplasts. These structures retained some cell-wall components but had large surface areas in which the cytoplasmic membrane was exposed. U.S. Pat. No. 5,169,772 discloses a method for purifying heparinase from bacteria comprising disrupting the envelope of the bacteria in an osmotically-stabilized medium, e.g., 20% sucrose solution using, e.g., EDTA, lysozyme, or an organic compound, releasing the non-heparinase-like proteins from the periplasmic space of the disrupted bacteria by exposing the bacteria to a low-ionic-strength buffer, and releasing the heparinase-like proteins by exposing the low-ionic-strength-washed bacteria to a buffered salt solution.

Many different modifications of these methods have been used on a wide range of expression systems with varying degrees of success (Joseph-Liazun et al. (1990) Gene, 86: 291-295; Carter et al. (1992) Bio/Technology, 10: 163-167). Efforts to induce recombinant cell culture to produce lysozyme have been reported. EP 0 155 189 discloses a means for inducing a recombinant cell culture to produce lysozymes, which would ordinarily be expected to kill such host cells by means of destroying or lysing the cell wall structure.

U.S. Pat. No. 4,595,658 discloses a method for facilitating externalization of proteins transported to the periplasmic space of E. coli. This method allows selective isolation of proteins that locate in the periplasm without the need for lysozyme treatment, mechanical grinding, or osmotic shock treatment of cells. U.S. Pat. No. 4,637,980 discloses producing a bacterial product by transforming a temperature-sensitive lysogen with a DNA molecule that codes, directly or indirectly, for the product, culturing the transformant under permissive conditions to express the gene product intracellularly, and externalizing the product by raising the temperature to induce phage-encoded functions. Asami et al. (1997) J. Ferment. and Bioeng., 83: 511-516 discloses synchronized disruption of E. coli cells by T4 phage infection, and Tanji et al. (1998) J. Ferment. and Bioeng., 85: 74-78 discloses controlled expression of lysis genes encoded in T4 phage for the gentle disruption of E. coli cells.

Upon cell lysis, genomic DNA leaks out of the cytoplasm into the medium and results in significant increase in fluid viscosity that can impede the sedimentation of solids in a centrifugal field. In the absence of shear forces such as those exerted during mechanical disruption to break down the DNA polymers, the slower sedimentation rate of solids through viscous fluid results in poor separation of solids and liquid during centrifugation. Other than mechanical shear force, there exist nucleolytic enzymes that degrade DNA polymer. In E. coli, the endogenous gene endA encodes for an endonuclease (molecular weight of the mature protein is approx. 24.5 kD) that is normally secreted to the periplasm and cleaves DNA into oligodeoxyribonucleotides in an endonucleolytic manner. It has been suggested that endA is relatively weakly expressed by E. coli (Wackemagel et al. (1995) Gene 154: 55-59).

In one embodiment, no additional disulfide-bond-promoting conditions or agents are required in order to recover disulfide-bond-containing identified polypeptide in active, soluble form from the host cell. In one embodiment, the transgenic polypeptide, polypeptide, protein, or fragment thereof has a folded intramolecular conformation in its active state. In one embodiment, the transgenic polypeptide, polypeptide, protein, or fragment contains at least one intramolecular disulfide bond in its active state; and perhaps up to 2, 4, 6, 8, 10, 12, 14, 16, 18, or 20 or more disulfide bonds.

The proteins produced using the methods of this invention may be isolated and purified to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, and others. For example, proteins having established molecular adhesion properties can be reversibly fused with a ligand. With the appropriate ligand, the protein can be selectively adsorbed to a purification column and then freed from the column in a relatively pure form. The fused protein is then removed by enzymatic activity. In addition, protein can be purified using immunoaffinity columns or Ni-NTA columns. General techniques are further described in, for example, R. Scopes, Protein Purification: Principles and Practice, Springer-Verlag: N.Y. (1982); Deutscher, Guide to Protein Purification, Academic Press (1990); U.S. Pat. No. 4,511,503; S. Roe, Protein Purification Techniques: A Practical Approach (Practical Approach Series), Oxford Press (2001); D. Bollag, et al., Protein Methods, Wiley-Lisa, Inc. (1996); AK Patra et al., Protein Expr Purif, 18(2): p/182-92 (2000); and R. Mukhija, et al., Gene 165(2): p. 303-6 (1995). See also, for example, Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QUIAGEN, Inc., Chatsworth, Calif.

Detection of the expressed protein is achieved by methods known in the art and include, for example, radioimmunoassays, Western blotting techniques or immunoprecipitation.

Alternatively, it is possible to purify the proteins or polypeptides of interest from the host periplasm. After lysis of the host cell, when the protein is exported into the periplasm of the host cell, the periplasmic fraction of the bacteria can be isolated by cold osmotic shock in addition to other methods known to those skilled in the art. To isolate targeted proteins from the periplasm, for example, the bacterial cells can be centrifuged to form a pellet. The pellet can be resuspended in a buffer containing 20% sucrose. To lyse the cells, the bacteria can be centrifuged and the pellet can be resuspended in ice-cold 5 mM MgSO₄ and kept in an ice bath for approximately 10 minutes. The cell suspension can be centrifuged and the supernatant decanted and saved. The targeted proteins present in the supernatant can be separated from the host proteins by standard separation techniques well known to those of skill in the art.

An initial salt fractionation can separate many of the unwanted host cell proteins (or proteins derived from the cell culture media) from the protein or polypeptide of interest. One such example can be ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of a protein or polypeptide of interest can be used to isolated it from proteins of greater and lesser size using ultrafiltration through membranes of different pore size (for example, Amicon or Millipore membranes). As a first step, the protein mixture can be ultrafiltered through a membrane with a pore size that has a lower molecular weight cut-off than the molecular weight of the protein of interest. The retentate of the ultrafiltration can then be ultrafiltered against a membrane with a molecular cut off greater than the molecular weight of the protein of interest. The protein or polypeptide of interest will pass through the membrane into the filtrate. The filtrate can then be chromatographed as described below.

The secreted proteins or polypeptides of interest can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

F. Proteins of Interest

The methods and compositions of the present invention are useful for producing high levels of properly processed protein or polypeptide of interest in a cell expression system. The protein or polypeptide of interest can be of any species and of any size. However, in certain embodiments, the protein or polypeptide of interest is a therapeutically useful protein or polypeptide. In some embodiments, the protein can be a mammalian protein, for example a human protein, and can be, for example, a growth factor, a cytokine, a chemokine or a blood protein. The protein or polypeptide of interest can be processed in a similar manner to the native protein or polypeptide. In certain embodiments, the protein or polypeptide does not include a secretion signal in the coding sequence. In certain embodiments, the protein or polypeptide of interest is less than 100 kD, less than 50 kD, or less than 30 kD in size. In certain embodiments, the protein or polypeptide of interest is a polypeptide of at least about 5, 10, 15, 20, 30, 40, 50 or 100 amino acids.

Extensive sequence information required for molecular genetics and genetic engineering techniques is widely publicly available. Access to complete nucleotide sequences of mammalian, as well as human, genes, cDNA sequences, amino acid sequences and genomes can be obtained from GenBank at the website //www.ncbi.nlm.nih.gov/Entrez. Additional information can also be obtained from GeneCards, an electronic encyclopedia integrating information about genes and their products and biomedical applications from the Weizmann Institute of Science Genome and Bioinformatics (bioinformatics.weizmann.ac.il/cards), nucleotide sequence information can be also obtained from the EMBL Nucleotide Sequence Database (www.ebi.ac.uk/embl/) or the DNA Databank or Japan (DDBJ, www.ddbi.nig.ac.ii/; additional sites for information on amino acid sequences include Georgetown's protein information resource website (www-nbrf.Reorgetown.edu/pirl) and Swiss-Prot (au.expasy.org/sprot/sprot-top.html).

Examples of proteins that can be expressed in this invention include molecules such as, e.g., renin, a growth hormone, including human growth hormone; bovine growth hormone; growth hormone releasing factor; parathyroid hormone; thyroid stimulating hormone; lipoproteins; α-1-antitrypsin; insulin A-chain; insulin B-chain; proinsulin; thrombopoietin; follicle stimulating hormone; calcitonin; luteinizing hormone; glucagon; clotting factors such as factor VIIIC, factor IX, tissue factor, and von Willebrands factor; anti-clotting factors such as Protein C; atrial naturietic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; a serum albumin such as human serum albumin; mullerian-inhibiting substance; relaxin A-chain; relaxin B-chain; prorelaxin; mouse gonadotropin-associated polypeptide; a microbial protein, such as beta-lactamase; Dnase; inhibin; activin; vascular endothelial growth factor (VEGF); receptors for hormones or growth factors; integrin; protein A or D; rheumatoid factors; a neurotrophic factor such as brain-derived neurotrophic factor (BDNF), neurotrophin-3, -4, -5, or -6 (NT-3, NT-4, NT-5, or NT-6), or a nerve growth factor such as NGF-β; cardiotrophins (cardiac hypertrophy factor) such as cardiotrophin-1 (CT-1); platelet-derived growth factor (PDGF); fibroblast growth factor such as aFGF and bFGF; epidermal growth factor (EGF); transforming growth factor (TGF) such as TGF-alpha and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; anti-HER-2 antibody; superoxide dismutase; T-cell receptors; surface membrane proteins; decay accelerating factor; viral antigen such as, for example, a portion of the AIDS envelope; transport proteins; homing receptors; addressins; regulatory proteins; antibodies; and fragments of any of the above-listed polypeptides.

In certain embodiments, the protein or polypeptide can be selected from IL-1, IL-1a, IL-1b, IL-2, IL-3, IL-4, IL-5, IL-6, IL-7, IL-8, IL-9, IL-10, IL-11, IL-12, IL-12elasti, IL-13, IL-15, IL-16, IL-18, IL-18BPa, IL-23, IL-24, VIP, erythropoietin, GM-CSF, G-CSF, M-CSF, platelet derived growth factor (PDGF), MSF, FLT-3 ligand, EGF, fibroblast growth factor (FGF; e.g., α-FGF (FGF-1), β-FGF (FGF-2), FGF-3, FGF-4, FGF-5, FGF-6, or FGF-7), insulin-like growth factors (e.g., IGF-1, IGF-2); tumor necrosis factors (e.g., TNF, Lymphotoxin), nerve growth factors (e.g., NGF), vascular endothelial growth factor (VEGF); interferons (e.g., IFN-α, IFN-β, IFN-γ); leukemia inhibitory factor (LIF); ciliary neurotrophic factor (CNTF); oncostatin M; stem cell factor (SCF); transforming growth factors (e.g., TGF-α, TGF-β1, TGF-β2, TGF-β3); TNF superfamily (e.g., LIGHT/TNFSF14, STALL-1/TNFSF13B (BLy5, BAFF, THANK), TNFalpha/TNFSF2 and TWEAK/TNFSF12); or chemokines (BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1AMCAF, MCP-3, MCP-4, MDC/STCP-1/ABCD-1, MIP-1 quadrature., MIP-1 quadrature., MIP-2.quadrature./GRO.quadrature., MIP-3.quadrature./Exodus/LARC, MIP-3/Exodus-3/ELC, MIP-4/PARC/DC-CK1, PF-4, RANTES, SDF1, TARC, or TECK).

In one embodiment of the present invention, the protein of interest can be a multi-subunit protein or polypeptide. Multisubunit proteins that can be expressed include homomeric and heteromeric proteins. The multisubunit proteins may include two or more subunits, that may be the same or different. For example, the protein may be a homomeric protein comprising 2, 3, 4, 5, 6, 7, 8, 9, 10, 11, 12 or more subunits. The protein also may be a heteromeric protein including 2, 3, 4, 5, 6, 7, 8, 9, 10, 11, 12, or more subunits. Exemplary multisubunit proteins include: receptors including ion channel receptors; extracellular matrix proteins including chondroitin; collagen; immunomodulators including MHC proteins, full chain antibodies, and antibody fragments; enzymes including RNA polymerases, and DNA polymerases; and membrane proteins.

In another embodiment, the protein of interest can be a blood protein. The blood proteins expressed in this embodiment include but are not limited to carrier proteins, such as albumin, including human and bovine albumin, transferrin, recombinant transferrin half-molecules, haptoglobin, fibrinogen and other coagulation factors, complement components, immunoglobulins, enzyme inhibitors, precursors of substances such as angiotensin and bradykinin, insulin, endothelin, and globulin, including alpha, beta, and gamma-globulin, and other types of proteins, polypeptides, and fragments thereof found primarily in the blood of mammals. The amino acid sequences for numerous blood proteins have been reported (see, S. S. Baldwin (1993) Comp. Biochem Physiol. 106b:203-218), including the amino acid sequence for human serum albumin (Lawn, L. M., et al. (1981) Nucleic Acids Research, 9: 6103-6114.) and human serum transferrin (Yang, F. et al. (1984) Proc. Natl. Acad. Sci. USA 81: 2752-2756).

In another embodiment, the protein of interest can be a recombinant enzyme or co-factor. The enzymes and co-factors expressed in this embodiment include but are not limited to aldolases, amine oxidases, amino acid oxidases, aspartases, B12 dependent enzymes, carboxypeptidases, carboxyesterases, carboxylyases, chemotrypsin, CoA requiring enzymes, cyanohydrin synthetases, cystathione synthases, decarboxylases, dehydrogenases, alcohol dehydrogenases, dehydratases, diaphorases, dioxygenases, enoate reductases, epoxide hydrases, fumerases, galactose oxidases, glucose isomerases, glucose oxidases, glycosyltrasferases, methyltransferases, nitrile hydrases, nucleoside phosphorylases, oxidoreductases, oxynitilases, peptidases, glycosyltrasferases, peroxidases, enzymes fused to a therapeutically active polypeptide, tissue plasminogen activator; urokinase, reptilase, streptokinase; catalase, superoxide dismutase; Dnase, amino acid hydrolases (e.g., asparaginase, amidohydrolases); carboxypeptidases; proteases, trypsin, pepsin, chymotrypsin, papain, bromelain, collagenase; neuramimidase; lactase, maltase, sucrase, and arabinofuranosidases.

In another embodiment, the protein of interest can be a single chain, Fab fragment and/or full chain antibody or fragments or portions thereof. A single-chain antibody can include the antigen-binding regions of antibodies on a single stably-folded polypeptide chain. Fab fragments can be a piece of a particular antibody. The Fab fragment can contain the antigen binding site. The Fab fragment can contain 2 chains: a light chain and a heavy chain fragment. These fragments can be linked via a linker or a disulfide bond.

The coding sequence for the protein or polypeptide of interest can be a native coding sequence for the target polypeptide, if available, but will more preferably be a coding sequence that has been selected, improved, or optimized for use in the selected expression host cell: for example, by synthesizing the gene to reflect the codon use bias of the host cell. Genetic code selection and codon frequency enhancement may be performed according to any of the various methods known to one of ordinary skill in the art, e.g., oligonucleotide-directed mutagenesis. Useful on-line InterNet resources to assist in this process include, e.g.: (1) the Codon Usage Database of the Kazusa DNA Research Institute (2-6-7 Kazusa-kamatari, Kisarazu, Chiba 292-0818 Japan) and available at www.kazusa.orjp/codon; and (2) the Genetic Codes tables available from the NCBI Taxonomy database at www.ncbi.nln.nih.gov/-Taxonomy/Utils/wprintgc.cgi?mode=c. For example, Pseudomonas species are reported as utilizing Genetic Code Translation Table 11 of the NCBI Taxonomy site, and at the Kazusa site as exhibiting the codon usage frequency of the table shown at www.kazusa.or.ip/codon/cgibin.

The gene(s) that result will have been constructed within or will be inserted into one or more vectors, which will then be transformed into the expression host cell. Nucleic acid or a polynucleotide said to be provided in an “expressible form” means nucleic acid or a polynucleotide that contains at least one gene that can be expressed by the selected expression host cell.

In certain embodiments, the protein of interest is, or is substantially homologous to, a native protein, such as a native mammalian or human protein. In these embodiments, the protein is not found in a concatameric form, but is linked only to a secretion signal and optionally a tag sequence for purification and/or recognition.

In other embodiments, the protein of interest is a protein that is active at a temperature from about 20 to about 42° C. In one embodiment, the protein is active at physiological temperatures and is inactivated when heated to high or extreme temperatures, such as temperatures over 65° C.

In other embodiments, the protein when produced also includes an additional targeting sequence, for example a sequence that targets the protein to the periplasm or to the extracellular medium. In one embodiment, the additional targeting sequence is operably linked to the carboxy-terminus of the protein. In another embodiment, the protein includes a secretion signal for an autotransporter, a two partner secretion system, a main terminal branch system or a fimbrial usher porin. See, for example, U.S. Patent Application Nos. 60/887,476 and 60/887,486, filed Jan. 31, 2007, herein incorporated by reference in their entireties).

The following examples are offered by way of illustration and not by way of limitation.

### EXPERIMENTAL EXAMPLES

### Construction of the COP-GFP-BspLEI Expression Plasmid

To facilitate ligation of a randomized RBS library fragment into a COP-GFP expression plasmid, the COP-GFP coding sequence was modified to incorporate a unique BspEI restriction site (5′ . . . TCCGGA . . . 3′, residues 33 through 38 of SEQ ID NO:10) beginning ten nucleotides downstream from the A nucleotide of the start codon (ATG). Primers RC-344 and RC-345 (Table 4) were used to amplify the COP-GFP coding sequence from pDOW2237 template DNA incorporating XbaI and XhoI restriction sites on the ends of the fragment. The RC-344 primer also produced the G12C silent mutation that resulted in the creation of a BspEI restriction site (FIG. 1). The PCR generated COP-GFP-BspEI fragment was then ligated into the XbaI-XhoI sites of expression plasmid pDOW1169 (dual lacO tac, pyrF+) to generate plasmid pDOW2260.

TABLE 4

| | | |
|---|---|---|
| Name | Sequence (5′ to 3′) | SEQ ID NO: |

| | | |
|---|---|---|
| RC-RBS | AATCTACTAGTNNNNNNNTCTAGAATGAGAGGATCCGGATCCCCCG | 10 |
| RC-344 | AATTTCTAGAATGAGAGGATCCGGATCCCCCGCCATGAAGAT | 11 |
| RC-345 | ATATCTCGAGTCAGGCGAATGCGATCGGGG | 12 |
| RC-348 | CGGGGGATCCGGATCCTCTCATTCTAGA | 13 |

### Construction of a Randomized Ribosome-Binding Site (RBS) Library

Oligonucleotides of 45 bp in length (RC-RBS) were generated containing SpeI, XbaI, and BspEI restriction sites with six bases of randomized nucleotides (A, T, C, or G) placed between the SpeI and XbaI restriction sites in order to randomize the AGGAGG sequence of the consensus RBS (SEQ ID NO: 1). A fill-in reaction was performed using primer RC-348 and the Pfu Turbo Hotstart PCR Master Mix to generate double-stranded fragments (FIG. 2). The fill-in reaction mixture (50 μL) contained 3.2 μM of RC-RBS and 6.4 μM of fill-in primer RC-348 and was treated for 2 min. at 95° C. followed by 1 min. at 68° C., and 10 min. at 72° C. The fill-in reaction was then purified using the QIAquick Nucleotide Removal Kit (Qiagen #28304) then sequentially digested with SpeI and BspEI. The digested fragments were then purified and concentrated using a Micron YM-10 centrifugal filter (Millipore #42407) and then ligated into SpeI and BspEI digested plasmid pDOW2260, which already contained the cloned COP-GFP reporter gene, to generate a plasmid library of alternative ribosome binding sites that can be screened for translational strength using COP-GFP as a reporter gene.

### Screen for RBS Sequences Producing a Range of COP-GFP Expression Levels

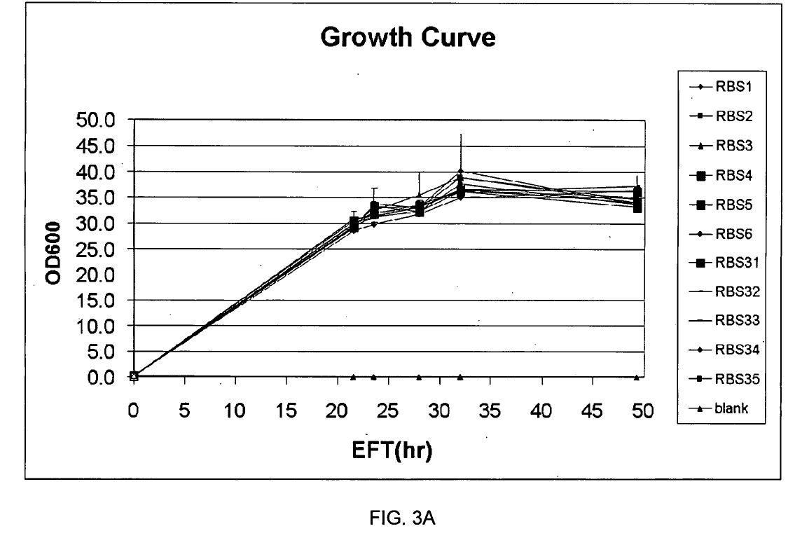

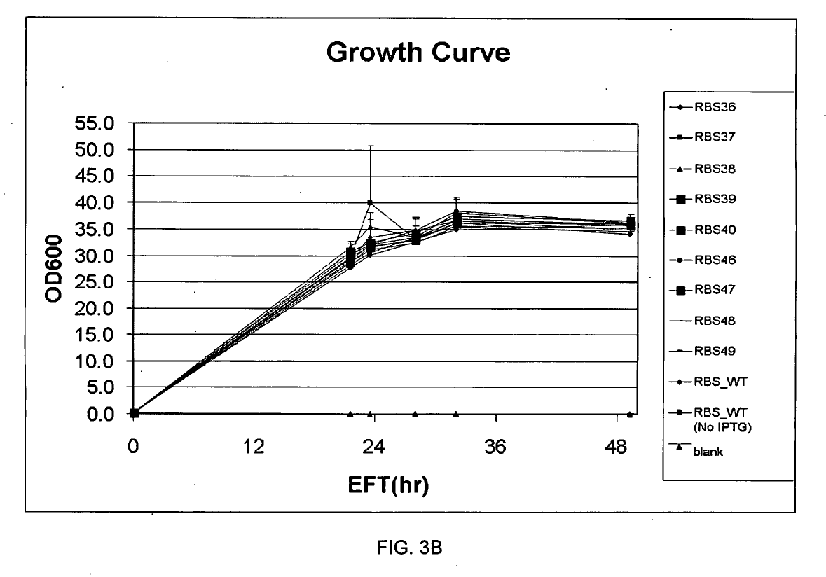

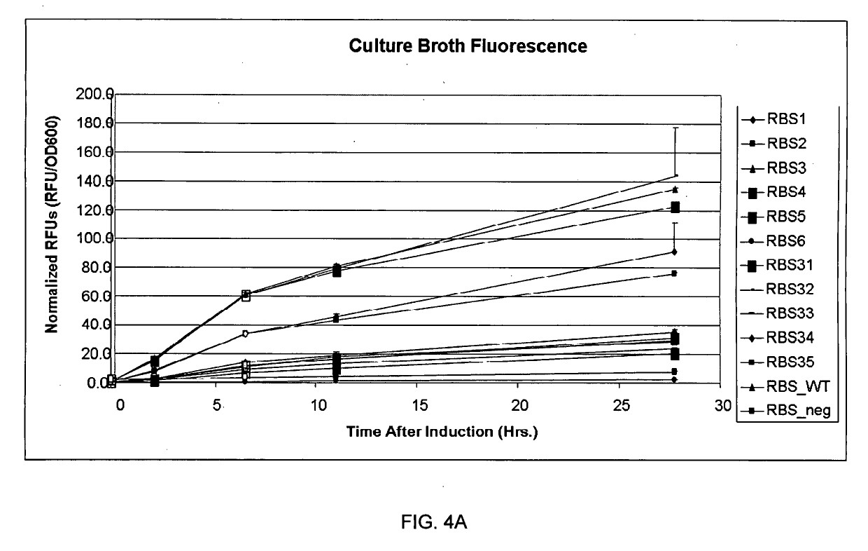

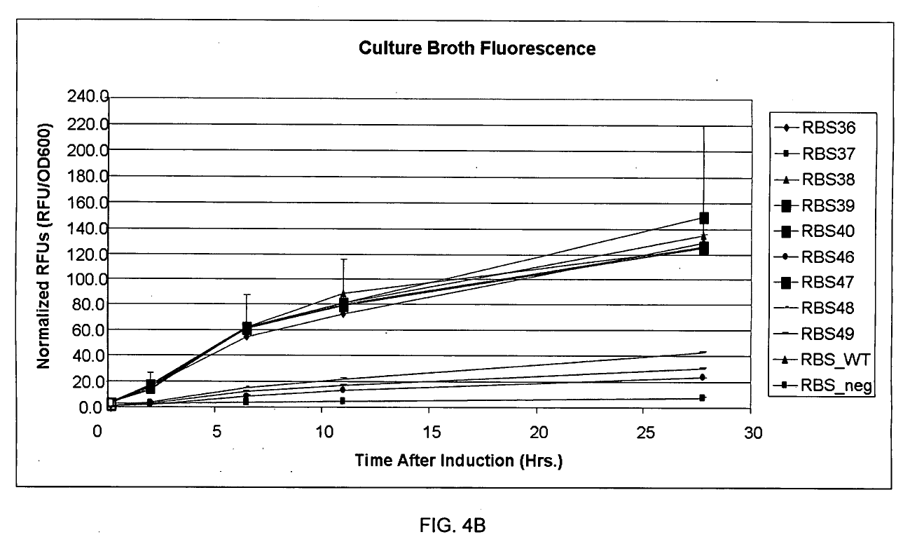

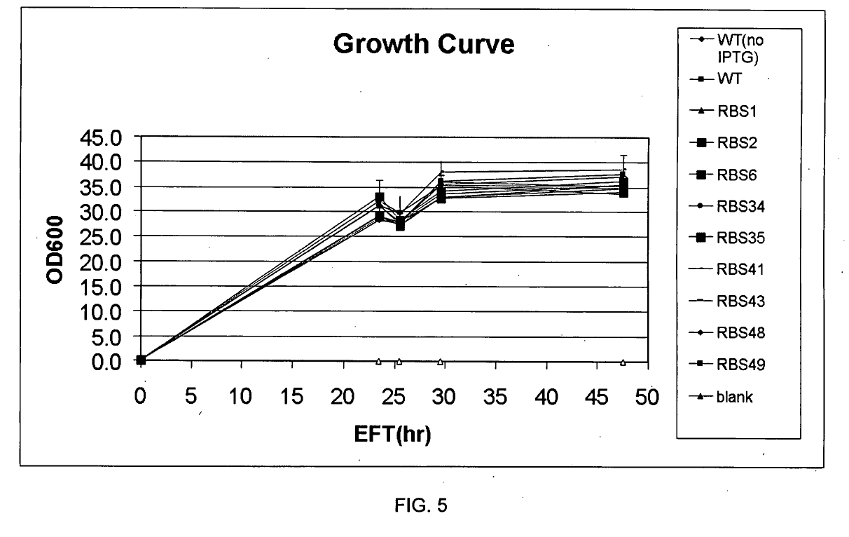

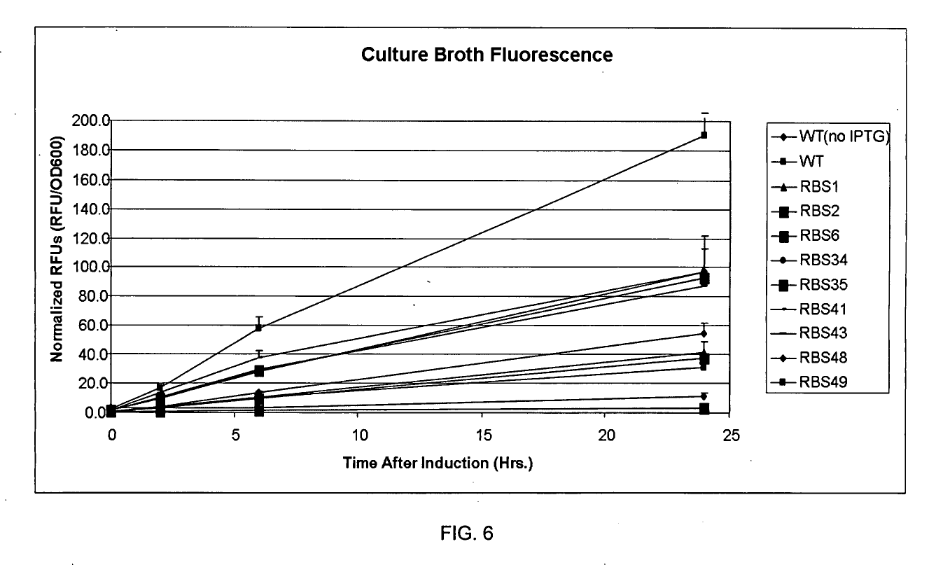

The randomized RBS plasmid library was electroporated into the P. fluorescens DC454 host strain and the transformed cells were then plated on to M9+1% glucose medium supplemented with 0.1 mM IPTG and incubated at 30° C. Colonies were visually screened for fluorescence from 30 hours (1 mm diameter) to approximately 72 hours (3 mm diameter) incubation by placing the transformation plates on a DARK READER™ transilluminator (Clare Chemical Research). Colonies exhibiting fluorescence were patched to plates and cultured overnight (16 hrs.) in 5 mL M9+1% glucose medium.
Comparison of COP-GFP Expression from RBS Plasmid Library Isolates
In order to compare COP-GFP expression levels from different RBS variant isolates, each isolate was grown in quadruplicate using HTP medium in the 96-well deep-well format using the DOW HTP medium and protocol. Following an initial growth phase, expression from the tac promoter was induced with 0.3 mM isopropyl-β-D-1-thiogalactopyranoside (IPTG). Cultures were sampled at the time of induction (I=0) and at 2, 6, and 24 hours after induction. Both the cell density (OD₆₀₀) and culture broth fluorescence (Spectramax Gemini plate reader; excitation—485 nm, emission—538 nm, bandpass—530 nm) of the samples were measured.
Comparison of COP-GFP Expression from RBS Library Isolates
In order to quantify COP-GFP expression from RBS variants, 20 isolates were grown using the 96-well HTP format, each in quadruplicate wells. As control, a consensus, or wild type RBS (AGGAGG, SEQ ID NO: 1) isolate was grown with and without 0.3 mM IPTG induction. While the growth pattern produced from all the isolates examined was fairly similar (FIGS. 3A and 3B), the culture broth fluorescence measurements produced a range of COP-GFP expression (FIGS. 4A and 4B). A second growth experiment was performed using eight select isolates with known RBS sequences representing the full range of COP expression along with the consensus RBS control. Two new isolates, RBS41 and RBS43, were added to the second experiment since these isolates yielded unique RBS sequences. While again, the growth pattern produced from all the isolates in the second growth experiment looked very similar (FIG. 5), the culture broth fluorescence measurements produced a range of COP-GFP expression (FIG. 6). The eight RBS variant sequences were ranked according to percentage of consensus RBS fluorescence measured at I=24 hours (averaged from quadruplicate culture wells). Each RBS variant was then placed into one of three general fluorescence ranks: High (“Hi”-100% Consensus RBS fluorescence), Medium (“Med”—46-51% of Consensus RBS fluorescence), and Low (“Lo”—16-29% Consensus RBS fluorescence) (Table 5).

TABLE 5

| | | | | | |
|---|---|---|---|---|---|
| | | | 1^{st} HTP | 2^{nd} HTP | 2^{nd} HTP |
| | | | 051201 | 060103 | 060103 |
| | | SEQ | COP % | COP % | Fluores- |
| COP+ | | ID | Consensus | Consensus | cence |
| isolate | RBS seq | NO: | @ I = 24 | @ I = 24 | Rank |

| | | | | | |
|---|---|---|---|---|---|
| Consensus | AGGAGG | 1 | 100 | 100 | High |
| RBS2 | GGAGCG | 2 | 66 | 49 | Med |
| RBS34 | GGAGCG | 2 | 79 | 51 | Med |
| RBS41 | AGGAGT | 3 | NA | 51 | Med |
| RBS43 | GGAGTG | 4 | NA | 46 | Med |
| RBS48 | GAGTAA | 5 | 22 | 29 | Low |
| RBS1 | AGAGAG | 6 | 21 | 22 | Low |
| RBS35 | AAGGCA | 7 | 19 | 20 | Low |
| RBS49 | CCGAAC | 8 | 0.02 | 16 | Low |

### Expression of Nef Using Varying Ribosome Binding Sites

Nef is a 206 amino acid protein encoded by HIV-1. It is expressed in the cytoplasm of the human cell, but can be membrane-bound through attachment to a myristol chain (a pathway that does not exist in bacteria) and is also found in an extracellular location (Macreadie, I. G., M. G. Lowe, et al. (1997) Biochem. Biophys. Res. Commun. 232(3): 707-711). It occurs in multiple forms that reflect its complex biological roles (Arold, S. T. and A. S. Baur (2001) Trends Biochem. Sci. 26(6): 356-363) including oligomers stabilized by disulfide bonds and noncovalent bonds (Kienzle, N., J. Freund, et al. (1993). Eur. J. Biochem. 214(2): 451-7).
The nef gene was cloned into pDOW1169, a P. fluorescens cytoplasmic expression vector, and in a nine-plasmid library that contained one of three signal sequences (Pbp, DsbA, or Azu) for directing Nef to the periplasm and one of three ribosome binding sites (selected from one high, one medium, and one low according to Table 5; “hi”=high; “me”=medium; and “lo”=low) to control the level of expression. All plasmids contained a Ptac promoter regulated by IPTG.
Strains were grown in quadruplicate in 96-well plates and induced by IPTG at 24 hr after inoculation; at I=24, cultures were normalized to OD₆₀₀=20, sonicated, and separated into soluble and insoluble fractions by centrifugation. The induction of Nef expression was well tolerated by the cell; strains expressing Nef achieved a final OD₆₀₀ between 40 and 55. The highest soluble expression detected for the nine periplasmic constructs was an average of 280 mg/L for the Azu-Hi construct.

### Expression of Pol-117 Using Varying Ribosome Binding Sites

Pol is an RNA-dependent DNA polymerase encoded by HIV-1. Upon infection of mammalian cells, the Gag-Pol preprotein is proteolytically cleaved into a Gag subunit and a Pol subunit (Jacks, T., M. Power, et al. (1988) Nature 331: 280-3.). The 117 kDa Pol subunit consists of multiple domains and is further proteolytically cleaved to result in a 66 kDa homodimer (p66/p66) containing the reverse transcriptase and RNAseH domains which is subsequently cleaved to form a p51/p66 heterodimer (Unge, T., H. Ahola, et al. (1990) AIDS Res. Hum. Retroviruses 6(11): 1297-303). The p66 homodimer has a 3D structure that is different than p51/p66 and is less active (Kew, Y., Q. Song, et al. (1994). J. Biol. Chem. 269(21): 15331-6).
The pol117 gene was designed for periplasmic expression using the nine-plasmid library described above. Periplasmic strains expressing Pol117 achieved a final OD₆₀₀ between 38 and 58. Using SDS-capillary electrophoresis (SDS-CGE), no protein was detected in the soluble fraction but substantial accumulation was found in the insoluble fraction. The highest insoluble accumulation (˜1.2 g/L) occurred with the Pbp-Hi and DsbA-Hi constructs, whereas less than half as much protein accumulation occurred when the lower strength ribosome binding site was used (Pbp-Me).

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for identifying an optimal ribosomal binding site (RBS) sequence for expression of a heterologous protein of interest comprising:
a) obtaining a library of oligonucleotides comprising variant RBS sequences, wherein said variants are obtained by fully randomizing the RBS at each position corresponding to SEQ ID NO: 1;
b) introducing said library of variant RBS sequences into an expression construct comprising a gene encoding the heterologous protein of interest to generate a library of expression constructs;
c) introducing said library of expression constructs into a population of a host cell of interest;
d) maintaining said cells under conditions sufficient for the expression of said protein of interest in at least one cell;
e) selecting the optimal population of cells in which the heterologous protein of interest is produced, wherein the protein produced by said optimal population of cells exhibits one or more of improved expression, improved activity, improved solubility, or improved translocation compared to protein produced by other populations generated in step (c); and,
f) obtaining the RBS sequence from the construct present in the population of cells selected in step (e).

2. The method of claim 1, wherein said RBS is fully randomized only at positions corresponding to positions 1 through 4 of SEQ ID NO: 1.

3. The method of claim 1, wherein said library of variant RBS sequences consists of SEQ ID NO:2, 3, 4, 5, 6, 7, and 8.

4. The method of claim 1, wherein said host cell is a bacterial host cell.

5. The method of claim 4, wherein said host cell is a Pseudomonad.

6. The method of claim 5, wherein said host cell is Pseudomonas fluorescens.

7. The method of claim 4, wherein said host cell is E. coli.

8. The method of claim 1, wherein said oligonucleotides comprise at least one restriction endonuclease cleavage site on the 3′ and the 5′ ends of said oligonucleotides.

9. The method of claim 1, wherein the translational efficiency of said optimal RBS sequence is at least 2-fold lower than the translational efficiency of the canonical RBS sequence.

10. The method of claim 9, wherein the translational efficiency of said optimal RBS sequence is 2-fold to 6-fold lower than the translational efficiency of the canonical RBS sequence.

11. The method of claim 1, wherein the cell is grown in a mineral salts media.

12. The method of claim 1, wherein the cell is grown at a high cell density.

13. The method of claim 12 wherein the cell is grown at a cell density of at least 20 g/L.

14. The method of claim 1, further comprising a step of purifying the heterologous protein.

15. The method of claim 14 wherein the heterologous protein is purified by affinity chromatography.

16. An isolated polynucleotide comprising an RBS sequence selected from the group consisting of SEQ ID NO:2, 3, 4, 5, 6, 7, and 8.

17. A vector comprising the isolated polynucleotide of claim 16.

18. The vector of claim 17 further comprising a polynucleotide encoding a protein or polypeptide of interest.

19. The vector of claim 18, wherein the protein or polypeptide of interest is derived from a eukaryotic organism.

20. The vector of claim 19, wherein the protein or polypeptide of interest is derived from a mammalian organism.

21. The vector of claim 17, wherein the vector further comprises a promoter.

22. The vector of claim 21 wherein the promoter is native to a bacterial host cell.

23. The vector of claim 21 wherein the promoter is not native to a bacterial host cell.

24. The vector of claim 21 wherein the promoter is native to E. coli.

25. The vector of claim 21, wherein the promoter is an inducible promoter.

26. The vector of claim 21, wherein the promoter is a lac promoter or a derivative of a lac promoter.

27. The vector of claim 18, wherein the polynucleotide encoding the protein or polypeptide of interest has been adjusted to reflect the codon preference of a host organism selected to express the polynucleotide.

28. A host cell comprising the vector of claim 17.

29. A kit comprising a library of oligonucleotides comprising variant RBS sequences, wherein said variants are obtained by fully randomizing the RBS at each position corresponding to SEQ ID NO: 1.

30. The kit of claim 29, wherein said RBS is fully randomized only at positions corresponding to positions 1 through 4 of SEQ ID NO: 1.

31. The kit of claim 29, wherein said library of variant RBS sequences consists of SEQ ID NO:2, 3, 4, 5, 6, 7, and 8.


SEQUENCE LISTING (13 sequences)

SEQ ID NO: 1 - DNA, 6 bp, Artificial Sequence
  canonical RBS sequence
aggagg

SEQ ID NO: 2 - DNA, 6 bp, Artificial Sequence
  RBS variant
ggagcg

SEQ ID NO: 3 - DNA, 6 bp, Artificial Sequence
  RBS variant
aggagt

SEQ ID NO: 4 - DNA, 6 bp, Artificial Sequence
  RBS variant
ggagtg

SEQ ID NO: 5 - DNA, 6 bp, Artificial Sequence
  RBS variant
gagtaa

SEQ ID NO: 6 - DNA, 6 bp, Artificial Sequence
  RBS variant
agagag

SEQ ID NO: 7 - DNA, 6 bp, Artificial Sequence
  RBS variant
aaggca

SEQ ID NO: 8 - DNA, 6 bp, Artificial Sequence
  RBS variant
ccgaac

SEQ ID NO: 9 - DNA, 240 bp, Artificial Sequence
  COP-GFP coding sequence in plasmid pDOW1169
tccgatgatcggtaaataccgatcaagcgcccaataccggcgattcaaggcaattgtgagcgctcacaatttattctgaaatgagctgttgacaattaatcatcggctcgtataatgtgtggaattgtgagcggataacaatttcacacaggaaacagaattttaatctactagtaggaggtctagaatgagaggatccggatcccccgccatgaagatcgagtgccgcatcaccggcac

SEQ ID NO: 10 - DNA, 45 bp, Artificial Sequence
  RC-RBS oligonucleotide primer
aatctactagtnnnnnntctagaatgagaggatccggatcccccg

SEQ ID NO: 11 - DNA, 42 bp, Artificial Sequence
  RC-344 oligonucleotide primer
aatttctagaatgagaggatccggatcccccgccatgaagat

SEQ ID NO: 12 - DNA, 30 bp, Artificial Sequence
  RC-345 oligonucleotide primer
atatctcgagtcaggcgaatgcgatcgggg

SEQ ID NO: 13 - DNA, 28 bp, Artificial Sequence
  RC-348 oligonucleotide primer
cgggggatccggatcctctcattctaga